(19) United States
(12) Patent Application Publication
(10) Pub. No.: US 2010/0029557 A1
(43) Pub. Date: Feb. 4, 2010

(54) ALPHA-LACTALBUMIN COMPOSITION

(76) Inventors: Jens Sigh, Frederiksberg (DK); Marianne Guldmann, Vedbaek (DK); Finn Matthiesen, Bronshoj (DK); Leif Kongerslev, Birkerod (DK)

Correspondence Address: NIXON & VANDERHYE, PC, 901 NORTH GLEBE ROAD, 11TH FLOOR, ARLINGTON, VA, 22203, US

(21) Appl. No.: 12/312,518

(22) PCT Filed: Nov. 15, 2007
(86) PCT No.: PCT/DK2007/050169

§ 371 Date: Aug. 24, 2009

(30) Foreign Application Priority Data
Nov. 17, 2006 (DK) PA 2006 01512

(51) Int. Cl. A61K 38/38; A61P 35/00; A61P 31/00

(52) U.S. Cl. 514/12

(57) ABSTRACT

The present invention relates to a pharmaceutical composition comprising monomeric alpha-lactalbumin complex, preferably LAC, which is an active complex of alpha-lactalbumin and a fatty acid or lipid with selective cytotoxic activity. The composition of the invention comprises insignificant amounts of oligomeric/multimeric alpha-lactalbumin complex, preferably LAC. Based on the selective cytotoxicity of the alpha-lactalbumin complex, preferably LAC composition such compositions are suitable for use in the manufacture of medicaments for use in therapy. Medicaments, comprising monomeric LAC are for use in the treatment of bacterial and viral infections and in particular cancer due to the selective cytotoxic activity. The application further relates to methods of producing a composition comprising monomeric alpha-lactalbumin complex, preferably LAC with cytotoxic activity.

All patent and non-patent references cited in the application, or in the present application, are also hereby incorporated by reference in their entirety.

### FIELD OF INVENTION

The present invention relates to a pharmaceutical composition comprising monomeric alpha-lactalbumin complex, preferably LAC, which is an active complex of alpha-lactalbumin and a fatty acid or lipid with selective cytotoxic activity. The composition of the invention comprises insignificant amounts of oligomeric/multimeric alpha-lactalbumin complex, preferably LAC. Based on the selective cytotoxicity of the alpha-lactalbumin complex, preferably LAC composition such compositions are suitable for use in the manufacture of medicaments for use in therapy. Medicaments, comprising monomeric LAC are for use in the treatment of bacterial and viral infections and in particular cancer due to the selective cytotoxic activity.

The application further relates to methods of producing a composition comprising monomeric alpha-lactalbumin complex, preferably LAC with cytotoxic activity

### BACKGROUND OF INVENTION

Monomeric alpha-lactalbumin (LA) is the most abundant protein in human milk whey. Mature monomeric alpha-lactalbumin consists of 123 amino acid residues (14.2 kDa) in many mammalian species. Human, bovine, equine, caprine, and camelide alpha-lactalbumin all consists of 123 amino acid residues, whereas porcine alpha-lactalbumin consists of 122 amino acids. Human, bovine, caprine and porcine alpha-lactalbumin also comprise a 19 amino acid leader sequence. This 14 KDa protein has been extensively characterised and the crystal structure has been resolved.

The crystal structure of alpha-lactalbumin has revealed that the protein consists of four alpha-helices and one triple stranded beta-sheet, which is found at the C-terminal end of the protein. The major alpha-helical domain contains amino acid 5-11, 23-34, 86-98, and the short alpha-helical segments; amino acid 18-20, 115-118. The beta-domain contains the triple-stranded anti-parallel sheet that consists of amino acids 40-50 and the short 76-82 helix.

Alpha-lactalbumin is a metalloprotein and comprises a high affinity Ca²⁺ binding site as well as several zinc binding sites. The high affinity Ca²⁺ binding site spans amino acid residues 77-89. In particular, residues 79, 82, 84, 87 and 88 appear to be involved in Ca²⁺ binding (Permyakov et al., α-Lactalbumin: structure and function. FEBS Letters 473 (2000) 269-274). Alpha-lactalbumin binds other physiologically significant cations such as Mg²⁺, Mn²⁺, Na⁺ and K⁺, which can compete with Ca²⁺ for the high affinity binding site.

The native monomer is the regulatory subunit of the lactose synthase complex, and alters the acceptor specificity of the galactosyl transferase from N-acetylglucosamine to glucose, with subsequent synthesis of lactose.

It has been shown that multimeric complexes of alpha-lactalbumin and a fatty acid or lipid may have cell killing abilities. A fraction from human milk containing an oligomeric complex was described as multimeric alpha-lactalbumin or MAL or HAM-LET (human a-lactalbumin made lethal to tumour cells) and was reported to have different biological properties to the monomeric form. It is a molecular complex that induces in vitro apoptosis selectively in tumour cells, but not in healthy differentiated cells.

The apoptotic activity of multimeric LA was discovered by serendipity. During a study regarding the effect of human milk on bacterial adherence, it was surprisingly discovered that human milk induced apoptosis in transformed and non-transformed immature cell lines. The apoptotic activity in human milk was isolated from a fraction of human milk casein obtained by precipitation at low pH, and was purified by ion exchange chromatography, eluting as a single peak after 1M NaCl. The elute was shown by spectroscopy to contain partially unfolded a-lactalbumin in an apo-like conformation (M. Svensson, et al, (1999) J Biol Chem, 274, 6388-96), with native-like secondary structure, but lacking specific tertiary packing of the side chains (M. Svensson, et al, (1999) J Biol Chem, 274, 6388-96). The link between apoptosis induction and the folding change was proven by deliberate conversion of native alpha-lactalbumin to the apoptosis inducing form (M. Svensson, et al., (2000) Proc Natl Acad Sci USA, 97, 4221-6). HAMLET was shown to bind to the surface of tumour cells, to translocate into the cytoplasm and to accumulate in cell nuclei, where it causes DNA fragmentation (M. Svensson, et al., (2000) Proc Natl Acad Sci USA, 97, 4221-6).

The oligomeric complex is reported as having therapeutic applications both in the field of antibiotic (WO96/04929) and cancer therapy (A. Hakansson et al., Proc. Natl. Acad. Sci. USA, (1995) 92, 8064-8068). In particular, the oligomeric form induces apoptotic cell death in cancer cells and immature cells, but not in healthy cells. These observations suggested that the protein acquires novel biological properties after conformational switching.

It is known that α-lactalbumin undergo conformational switching when exposed to low pH. The A state or molten globule state has native secondary structure, but less well defined tertiary structure than the native state. Similar states of a-lactalbumin can form also at neutral pH, upon removal of the tightly bound Ca²⁺ ion, reduction of disulphide bonds or at elevated temperatures.

It has also been found that other reagents and specifically lipid such as oleic acid are useful in the conversion of human a-lactalbumin to HAMLET. In particular, it has been reported previously that oleic acid (C18:1:9cis) is required for HAMLET production (M. Svensson, et al., (2000) Proc Natl Acad Sci USA, 97, 4221-6).

Binding of Ca²⁺ to a single very high affinity Ca²⁺ binding site is important for the protein to maintain a native conformation. The high affinity Ca²⁺ binding site is 100% conserved across many mammalian species including human, bovine, equine, porcine, caprine and camelide alpha-lactalbumin. Five of the seven oxygens that ligate the Ca²⁺ are contributed by side chain carboxylates of Asp residues at positions 82, 87 and 88 and by carbonyl oxygens of Lys 79 and Asp 84, and two water molecules supply the remaining ligands. The bound Ca²⁺ brings the α-helical region and the sheet in close proximity, and two disulfide bonds flanking the Ca²⁺ binding site, make this part of the molecule fairly inflexible. Binding of other cations, such as Mg²⁺, Mn²⁺, Na⁺ and K⁺ also cause conformational changes in alpha-lactalbumin although these are smaller than for the binding of Ca²⁺.

Conversion of human alpha-lactalbumin to LAC with apoptotic activity, has previously been found to require both a conformational or folding change, the presence of a fatty acid or a lipid and oligomerization. The conformational or folding change is conveniently effected by removal of calcium ions, or by using a variant without calcium ions. However, once the change has been effected, the presence of calcium or a functional calcium binding site does not result in any loss of activity. The oligomeric complex is reported as having therapeutic applications both in the field of antibiotic (WO96/04929) and cancer therapy (A. Hakansson et al., Proc. Natl. Acad. Sci. USA, (1995) 92, 8064-8068). In particular, the oligomeric form of LAC may induce apoptotic cell death in cancer cells and immature cells, but not (or only to a low extend) in mature, healthy cells. These observations suggested that the protein acquires novel biological properties when forming an active complex with a fatty acid or a lipid. Thus, reagents such as fatty acids or lipids, such as oleic acid, may be useful in the conversion of LA to LAC

Previously it has been thought that the cytotoxic activity against cancer cells and immature cells was a feature only of multimeric human LAC (A. Hakansson et al., Proc. Natl. Acad. Sci. USA, (1995) 92, 8064-8068, M. Svensson et al., JBC (1999) 274, 6388-6396, A. Hakansson et al. Molecular Microbiology (2000)35, 589-600). Using SDS-PAGE and MALDI-MS Hakansson et al (2000) analyzed a composition comprising both monomeric and oligomeric LAC. It was determined that the active cytotoxic fraction contained oligomeric forms of LAC and the cell killing activity of LAC was attributed to the oligomeric form only (A. Hakansson et al. Molecular Microbiology (2000)35, 589-600).

Xu et al (2005) showed in two separate papers that commercially available bovine alpha-lactalbumin and alpha-lactalbumin purified from cow's milk could be converted to a form that showed potent inhibition of cell proliferation and could induce cell death (Xu et al., Biosci. Biotechnol. Biochem. (2005)69, 1082-1089 and Xu et al., Biosci. Biotechnol. Biochem. (2005)69, 1189-1192). Only multimeric forms of alpha-lactalbumin exhibited these cytotoxic biological activities; the monomeric form did not exhibit any cell killing activity (Xu et al., Biosci. Biotechnol. Biochem. (2005)₆₉, 1082-1089 and Xu et al., Biosci. Biotechnol. Biochem. (2005)69, 1189-1192).

The oligomeric complex is reported as having therapeutic applications both in the field of antibiotic (WO96/04929) and cancer therapy (A. Hakansson et al., Proc. Natl. Acad. Sci. USA, (1995) 92, 8064-8068). In particular, the oligomeric form of LAC may induce apoptotic cell death in cancer cells and immature cells, but not (or only to a low extend) in mature, healthy cells. These observations suggested that the protein acquires novel biological properties when forming an active complex with a fatty acid or a lipid. Thus, reagents such as fatty acids or lipids, such as oleic acid, may be useful in the conversion of LA to LAC

### SUMMARY OF INVENTION

The present invention relates to the finding that monomeric LAC may comprise biological activities similar to multimeric LAC, including the selective cytotoxic activity, such as the cytotoxic activity towards cancer cells and immature cells.

An aspect of the invention relates to a pharmaceutical composition comprising a monomeric LAC, which is a complex of alpha-lactalbumin and a fatty acid or a lipid, said alpha-lactalbumin being human alpha-lactalbumin or bovine alpha-lactalbumin or a functional equivalent thereof, wherein the composition comprises at least 50% by weight of monomeric LAC.

Preferably the monomeric LAC composition has a selective cytotoxic activity, where the potency measured as LD50 is less than 0.1 mg/ml.

In a preferred embodiment the alpha-lactalbumin is bovine alpha-lactalbumin identified by SEQ ID NO 1. In another embodiment the alpha-lactalbumin is human alpha-lactalbumin identified by SEQ ID NO 2.

In a further aspect the invention relates to a method of producing a composition comprising monomeric LAC, which is a complex of alpha-lactalbumin and a fatty acid or a lipid, said alpha-lactalbumin being alpha-lactalbumin of SEQ ID NO:1 or SEQ ID NO:2 or a functional equivalent thereof, comprising a sequence at least 70% identical thereto, wherein the composition comprises at least 50% by weight of monomeric LAC comprising the steps of:
a. obtaining an alpha-lactalbumin composition comprising at least 95% by weight of monomeric alpha-lactalbumin,
b. conversion of said alpha-lactalbumin to LAC
   i. by release of calcium from said alpha-lactalbumin and
   ii. binding of fatty acid or lipid to said alpha-lactalbumin, and
c. purification of LAC.

In other equally preferred embodiments LAC may be produced according to the methods described in Examples 3 and 4 of Danish Patent Application PA 2007 0693.

In a yet further aspect the invention relates to the use of a composition comprising monomeric LAC, which is a complex of alpha-lactalbumin and a fatty acid or a lipid, said alpha-lactalbumin being alpha-lactalbumin of SEQ ID NO:1 or SEQ ID NO:2 or a functional equivalent thereof, comprising a sequence at least 70% identical thereto, wherein the composition comprises at least 50% by weight of monomeric LAC for the preparation of a medicament.

A subsequent aspect of the invention relates to a method of treatment comprising administering a medicament comprising:
i. a pharmaceutical composition comprising monomeric LAC, which is a complex of alpha-lactalbumin LA and a fatty acid or a lipid, said alpha-lactalbumin being of SEQ ID NO:1 or SEQ ID NO:2 or a functional equivalent thereof, comprising a sequence at least 70% identical thereto, wherein the composition comprises at least 50% by weight of monomeric LAC and
ii. pharmaceutical excipients.

When used herein:

The term “Alpha-lactalbumin” as used herein has the meaning of the alpha-lactalbumin polypeptide independent of the tertiary structure of the polypeptide. The sequences of bovine and human alpha-lactalbumin are defined by SEQ ID NO 1 and SEQ ID NO 2 respectively. 1A shows a sequence alignment of human and bovine alpha-lactalbumin. Thus human alpha-lactalbumin is any polypeptide of the sequence SEQ ID NO 2 with any tertiary structure. Similarly, bovine alpha-lactalbumin is any polypeptide of the sequence SEQ ID NO 1 with any tertiary structure.

The term “LA” as used herein has the meaning of the alpha-lactalbumin polypeptide preferably in the native tertiary structure and preferably with calcium bound to the high affinity calcium-binding site. LA is not in complex with any fatty acids or lipids and does preferably not have cell killing abilities. The terms “hLA” and “bLA” as used herein have the meaning of human LA and bovine LA, respectively.

A-state of alpha-lactalbumin has the meaning of partially folded state of alpha-lactalbumin adopted for example when dissolved at low pH, whereas the apo-state is the partially folded state alpha-lactalbumin adopted for example upon removal of the protein bound calcium at neutral pH and low salt concentration.

The term “LAC” as used herein has the meaning of an active complex of alpha-lactalbumin and a fatty acid or a lipid. By “active” is meant that the complex has capacity of apoptosis induction (see more details herein below in the section “Alpha-Lactalbumin”). The terms hLAC and bLAC as used herein has the meaning of human LAC and bovine LAC, respectively. Preferably, LAC has cell killing activity which is less than 15 μg/100.000 cells in 70 μl. In a preferred embodiment the LD50 is less than 10 μg/100.000 cells in 70 μl. The alpha-lactalbumin composition according to the invention more preferable has a cytotoxic activity measured as LD50 of less than 5 μg/100.000 cells. Most preferred is a composition wherein the cytotoxic activity measured as LD50 is 1-5 μg/100.000 cells in 70 μl.

### DETAILED DESCRIPTION OF THE INVENTION

The applicants herein describe a composition comprising alpha-lactalbumin complex, preferably LAC with a predominant content of monomeric LAC in comparison to the content of multimeric or oligomeric LAC.

Monomeric alpha-lactalbumin has a molecular weight of approximately 14 kDa. Monomers of alpha-lactalbumin can multimerize or oligomerize to form higher molecular weight molecules, for example when the monomers are passed over an ion exchange column (therapy (A. Hakansson et al., Proc. Natl. Acad. Sci. USA, (1995) 92, 8064-8068). These multimeric forms were proven to have selective cytotoxic activities (A. Hakansson et al., Proc. Natl. Acad. Sci. USA, (1995) 92, 8064-8068).

Similarly, a monomeric alpha-lactalbumin complex according to the invention preferably consists of monomeric alpha-lactalbumin and lipid or fatty acid.

In general, a monomeric alpha-lactalbumin molecule according to the invention contains only one alpha-lactalbumin polypeptide, such as any of the alpha-lactalbumin polypeptides described herein below. Preferably a monomeric alpha-lactalbumin molecule according to the invention has a molecular weight of in the range of 14 to 15 kDa. In general, a dimer of alpha-lactalbumin contains exactly two alpha-lactalbumin polypeptides, such as any of the alpha-lactalbumin polypeptides described herein below. Preferably, a dimer of alpha-lactalbumin has a molecular weight of in the range of 28-30 kDa. In general, a trimer of alpha-lactalbumin contains exactly three alpha-lactalbumin polypeptides, such as any of the alpha-lactalbumin polypeptides described herein below. Preferably, a trimer has a molecular weight of in the range of 42 to 45 kDa. Similar applies for higher oligomers.

The content of LAC monomers and oligomers is calculated by weight, i.e. the mass of the protein complexes. In an example wherein the relative content of alpha-lactalbumin molecules is as follows, a composition comprising 6 monomeric alpha-lactalbumin, 1 dimeric alpha-lactalbumin and 1 trimeric alpha-lactalbumin, the content of monomeric alpha-lactalbumin is 6×14/(6×14+2×14+3×14)×100%=84/154×100%=54.5%

The alpha-lactalbumin composition, preferably LAC according to the invention, comprises more than 50% (weight %) monomeric LAC, such as more than 60%, such as more than 70%, preferably more than 80% or more than 90% by weight of monomeric LAC, preferably more than 95% by weight of monomeric LAC.

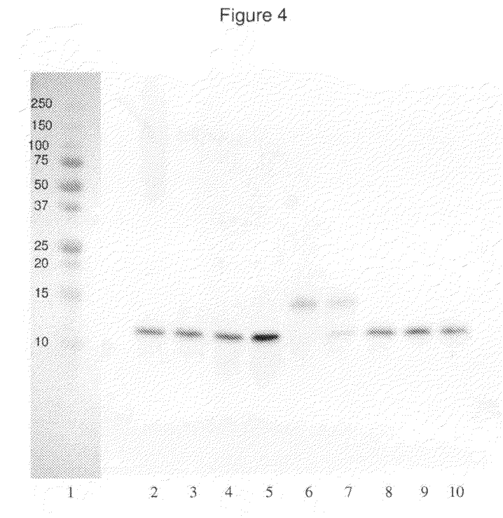

In particular preferred embodiment the composition comprises more than 96% (weight %) monomeric alpha-lactalbumin, preferably LAC, more preferably more than 97%, or more than 98%, most preferably more than 99% of monomeric alpha-lactalbumin, preferably LAC, thus the composition is preferably an essentially pure monomeric alpha-lactalbumin, preferably LAC composition comprising an insignificant amount of multimeric or oligomeric alpha-lactalbumin, preferably LAC. The alpha-lactalbumin complex composition described in example 1, and characterised by the FIGS. 4B and 4, shows a composition wherein the content of multimeric or oligomeric alpha-lactalbumin, preferably LAC is not detectable by either size exclusion chromatography (SEC) or by western blotting.

It is preferred that the amount of multimeric or oligomeric alpha-lactalbumin, preferably LAC in the composition is below the level of detection by a method such as PAGE or immunoblotting.

An aspect of the invention relates to a composition comprising monomeric alpha-lactalbumin, preferably LAC, which is a complex of alpha-lactalbumin and a fatty acid or a lipid, said alpha-lactalbumin being bovine or human alpha-lactalbumin or a functional equivalent thereof, wherein the composition comprises at least 95% by weight of monomeric LAC. In a preferred embodiment said alpha-lactalbumin is bovine alpha-lactalbumin.

The wild-type bovine alpha-lactalbumin i.e. the naturally occurring non-mutated version of the protein is identified as SEQ ID NO: 1 and the wild-type human alpha-lactalbumin i.e. the naturally occurring non-mutated version of the protein is identified as SEQ ID NO: 2 and. The present invention also covers functional homologues of alpha-lactalbumin comprising a sequence identity of at least 70% to SEQ ID NO: 1 or comprising a sequence identity of at least 70% to SEQ ID NO:2 as well as an active complex of functional homologues of alpha-lactalbumin with a fatty acid or a lipid. The wild-type bovine alpha-lactalbumin including the leader sequence i.e. the naturally occurring non-mutated version of the protein including the 19 amino acid leader sequence is identified as SEQ ID NO: 3 and the wild-type human alpha-lactalbumin including the leader sequence i.e. the naturally occurring non-mutated version of the protein including the 19 amino acid leader sequence is identified as SEQ ID NO: 4.

A functional homologue can be defined as alpha-lactalbumin that differs in sequence from the wild-type alpha-lactalbumin, such as wild-type human alpha-lactalbumin or wild-type bovine lactalbumin, but is still functionally competent. A functional homologue may be a mutated version or an alternative splice variant of the wild-type alpha-lactalbumin. In another aspect functional homologues of alpha-lactalbumin are defined as described herein below. A functional homologue may be, but is not limited to, a recombinant version of alpha-lactalbumin with one or more mutations and/or one or more sequence deletions and/or additions introduced ex vivo.

In preferred embodiments of the invention, the alpha-lactalbumin may be human or bovine alpha-lactalbumin, wherein the alpha-lactalbumin is either naturally occurring milk alpha-lactalbumin or the alpha-lactalbumin has been recombinantly produced.

### Alpha-Lactalbumin

Alpha-lactalbumin is as described in the background section highly abundant in milk. The sequence of alpha-lactalbumin from different mammal species is well conserved. Sequences from rodents (mouse, rat, rabbit, guinea pig), primates, cats and dogs show a high degree of identity. The amino acid sequence from equine, caprine, bovine, porcine and humans show approximately 75-95% identity (Pettersson, Jenny, BBRC 345 (2006) 260-270).

Alpha-lactalbumin from any species, preferably any mammalian species, may according to the invention be used for production of monomeric alpha-lactalbumin, preferably LAC. For the present invention alpha-lactalbumin from any species different from bovine or human species is considered functional equivalents (see below) of bovine or human alpha-lactalbumin. Alpha-lactalbumin is evolutionary related to and share around 35 to 40% of sequence homology as well as the positions of the four disulfide bonds with lysozyme C.

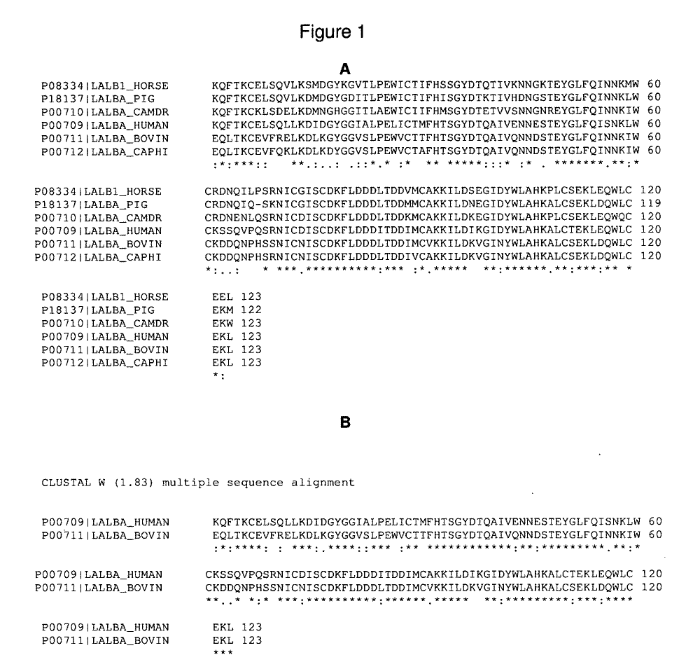

In an embodiment of the invention the functional equivalent of bovine or human alpha-lactalbumin is selected from the group consisting of equine, caprine, bovine, camelide and porcine. In a most preferred embodiment the alpha-lactalbumin is bovine. FIG. 1B shows an alignment of the protein sequences of bovine and human alpha-lactalbumin.

Human wild-type alpha-lactalbumin is identified as SEQ ID NO: 2 and bovine wild-type alpha-lactalbumin is identified as SEQ ID NO: 1. In one preferred embodiment of the invention alpha-lactalbumin is bovine alpha-lactalbumin and in another embodiment of the invention alpha-lactalbumin is human alpha-lactalbumin. In a more preferred embodiment alpha-lactalbumin is bovine wild-type alpha-lactalbumin as identified by SEQ ID NO: 1 and in another preferred embodiment of the invention alpha-tactalbumin is human alpha-lactalbumin as identified by SEQ ID NO: 2.

In another preferred embodiment alpha-lactalbumin is recombinant wild type human alpha-lactalbumin and in an equally preferred embodiment of the invention alpha-lactalbumin is recombinant wild type bovine alpha-lactalbumin. Alpha-lactalbumin variants include any form of alpha-lactalbumin known to a person skilled in the art and any functional homologue thereof. For example, alpha-lactalbumin variants include splice variants and allelic variants and single nucleotide polymorphisms.

A functional homologue of alpha-lactalbumin may be any protein that exhibits at least some sequence identity with SEQ ID NO: 1 or SEQ ID NO: 2, and when complexed with a fatty acid or a lipid shares one or more functions with LAC, such as the capacity of apoptosis induction (see more details herein below).

The capacity of LAC of induction of apoptosis can for example be measured as described in Danish patent application PA 2007 0693 or as in Example: Cytotocicity of monomeric alpha-lactalbumin composition. The capacity of alpha-lactalbumin of DNA fragmentation can be visualised as described in (Pettersson, Jenny, BBRC 345 (2006) 260-270) for example with ethidium bromide using a 305 nm UV-light source. Histone binding activity of alpha-lactalbumin, which is a function of wild type LAC can be measured as described in Danish patent application PA 2007 0693

Alpha-lactalbumin to be used with the present invention may be derived from any suitable source, for example alpha-lactalbumin may be naturally occurring alpha-lactalbumin or alpha-lactalbumin may be recombinantly produced alpha-lactalbumin as described in detail herein below. In a preferred embodiment alpha-lactalbumin is human alpha-lactalbumin purified from human milk and in another equally preferred embodiment alpha-lactalbumin is bovine alpha-lactalbumin purified from bovine milk.

In a preferred embodiment alpha-lactalbumin is recombinant bovine alpha-lactalbumin. In a more preferred embodiment alpha-lactalbumin is recombinant bovine wild-type alpha-lactalbumin.

### Functional Equivalents of Alpha-Lactalbumin

In one preferred embodiment of the invention alpha-lactalbumin is bovine alpha-lactalbumin, in a more preferred embodiment alpha-lactalbumin is bovine wild-type alpha-lactalbumin as identified by SEQ ID NO: 1. In a very preferred embodiment alpha-lactalbumin is recombinant wild type human alpha-lactalbumin. In another preferred embodiment of the invention alpha-lactalbumin is human alpha-lactalbumin, in a more preferred embodiment alpha-lactalbumin is human wild-type alpha-lactalbumin as identified by SEQ ID NO: 2. In a very preferred embodiment alpha-lactalbumin is recombinant wild type bovine alpha-lactalbumin.

It is evident from the above that a reasonable number of modifications or alterations of the bovine or human alpha-lactalbumin sequence does not interfere with the activity of the alpha-lactalbumin molecule according to the invention. Such alpha-lactalbumin molecules are herein referred to as functional equivalents of bovine or human alpha-lactalbumin, and may be such as variants and fragments of native bovine or human alpha-lactalbumin as described here below.

A functional homologue of alpha-lactalbumin may be any protein that exhibits at least some sequence identity with SEQ ID NO. 1 or SEQ ID NO.2 and shares one or more functions with alpha-lactalbumin, such as:
Acting as a co-factor in the synthesis of lactose
Exhibiting cell killing activity when converted to LAC
Capability to induce apoptosis when converted to LAC
Histone binding activity when converted to LAC

Several methods may be used to determine whether LAC has cell killing activity.

Several methods may be used to determine whether LAC has histone binding activity.

Preferably the functional homologue exhibits at least some sequence identity with SEQ ID NO. 1 or SEQ ID NO.2 and has cell killing abilities.

Preferably, evolutionary conservation between alpha-lactalbumin of different closely related species, e.g. assessed by sequence alignment, can be used to pinpoint the degree of evolutionary pressure on individual residues. Preferably, alpha-lactalbumin sequences are compared between species where alpha-lactalbumin function is conserved, for example but not limited to mammals including rodents, monkeys and apes. Residues under high selective pressure are more likely to represent essential amino acids that cannot easily be substituted than residues that change between species. For example, such an alignment may be performed using ClustalW from EBML-EBI comparing porcine alpha-lactalbumin and human alpha-lactalbumin (FIG. 1 A). It is evident from the above that a reasonable number of modifications or alterations of the bovine or human alpha-lactalbumin sequence does not interfere with the activity of the alpha-lactalbumin molecule according to the invention. Such alpha-lactalbumin molecules are herein referred to as functional equivalents of bovine or human alpha-lactalbumin, and may be such as variants and fragments of native bovine or human alpha-lactalbumin as described here below.

Functional assays can for example be used in order to determine if alpha-lactalbumin function is conserved. Functional assays known to a skilled person can be used to verify the functional conservation of uncomplexed alpha-lactalbumin. Such functional assays determine the ability of alpha-lactalbumin to act as a regulatory subunit of the lactose synthase complex in the production of lactose.

Functional assays known to a skilled person can be used to verify the functional conservation of alpha-lactalbumin in complex with a fatty acid or a lipid. Functional assays for evaluating alpha-lactalbumin function known to persons skilled in the art include, but are not limited to, assays described herein above and in Danish patent application PA 2007 0693.

As used herein the expression “variant” refers to polypeptides or proteins which are homologous to the basic protein, which is suitably bovine or human alpha-lactalbumin, but which differs from the base sequence from which they are derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide. FIG. 1A shows an alignment of the protein sequences of bovine, human, equine, caprine, bovine, camelide and porcine alpha-lactalbumin wherein identical residues (“*”) and residues with conservative (“:”) and semi-conservative (“.”) substitutions are marked.

Accordingly, in one embodiment of the invention it is preferred that functional homologues of alpha-lactalbumin comprises a sequence with high sequence identity to SEQ ID NO: 1 or SEQ ID NO:2, wherein none of the conserved residues marked with “*” in FIG. 1A are substituted. It is furthermore preferred within this embodiment that the residues marked with “:” in FIG. 1A are either not substituted or only substituted by conservative substitution, more preferably by substitution with an amino acid with a high level of similarity as defined herein below.

Thus in one embodiment it is preferred that functional homologues of bovine alpha-lactalbumin have a sequence with high sequence identity to SEQ ID NO: 1, wherein residues E1, L3, E7, V8, L15, Y18, V21, S22, V27, Q39, A40, I41, N44, 159, K62, Q65, I85, M90, N102, S112, D116, K122 are either not substituted or substituted only by conservative substitution, more preferably substituted only an amino acid with a high level of similarity as defined herein below.

It is even further preferred within the present invention that functional homologues of alpha-lactalbumin have a sequence with high sequence identity to SEQ ID NO:1 or SEQ ID NO: 2, wherein residues marked with “.” in FIG. 1A are either not substituted or are only substituted by conservative substitutions, such as with amino acids with lower levels or high level of similarity as defined herein below. Accordingly, it is preferred that functional homologues of bovine alpha-lactalbumin have a sequence with high sequence identity to SEQ ID NO: 1, wherein residues D14, K16, G17, G20, P24, S47, N56, D63, D64, N74, V92, and A109 are either not substituted or only substituted by conservative substitutions, such as with amino acids with lower level or high level of similarity as defined herein below.

It is also comprised within the present invention that functional homologues of alpha-lactalbumin may have a sequence with high sequence identity to SEQ ID NO: 1 or SEQ ID NO: 2, wherein the unmarkes residues in FIG. 1A may be substituted with any other amino acid. Thus, functional homolgous of human alpha-lactalbumin may have a sequence with high sequence identity to SEQ ID NO: 1, wherein residues F9, R10, E11, G19, W25, T29, T30, T33, Q43, D46, T48, N66, P67, H68, S70, I89, K98, V99, L118, and L123 are either not substituted or substituted with any other amino acid.

A person skilled in the art will know how to make and assess ‘conservative’ amino acid substitutions, by which one amino acid is substituted for another with one or more shared chemical and/or physical characteristics. Conservative amino acid substitutions are less likely to affect the functionality of the protein. Amino acids may be grouped according to shared characteristics. A conservative amino acid substitution is a substitution of one amino acid within a predetermined group of amino acids for another amino acid within the same group, wherein the amino acids within a predetermined groups exhibit similar or substantially similar characteristics.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine, a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

Within the meaning of the term “conservative amino acid substitution” as applied herein, one amino acid may be substituted for another within the groups of amino acids indicated herein below:

### Lower Levels of Similarity:

### Polarity:

i) Amino acids having polar side chains (Asp, Glu, Lys, Arg, His, Asn, Gln, Ser, Thr, Tyr, and Cys)
ii) Amino acids having non-polar side chains (Gly, Ala, Val, Leu, Ile, Phe, Trp, Pro, and Met)

### Hydrophilic or Hydrophobic:

iii) Hydrophobic amino acids (Ala, Cys, Gly, Ile, Leu, Met, Phe, Pro, Trp, Tyr, Val)
iv) Hydrophilic amino acids (Arg, Ser, Thr, Asn, Asp, Gln, Glu, His, Lys)

### Charges:

v) Neutral amino acids (Ala, Asn, Cys, Gln, Gly, Ile, Leu, Met, Phe, Pro, Ser, Thr, Trp, Tyr, Val)
vi) Basic amino acids (Arg, His, Lys)
vii) Acidic amino acids ((asp, Glu)

### High Level of Similarity:

viii) Acidic amino acids and their amides (Gln, Asn, Glu, Asp)
ix) Amino acids having aliphatic side chains (Gly, Ala Val, Leu, Ile)
x) Amino acids having aromatic side chains (Phe, Tyr, Trp)
xi) Amino acids having basic side chains (Lys, Arg, His)
xii) Amino acids having hydroxy side chains (Ser, Thr)
xiii) Amino acids having sulphor-containing side chains (Cys, Met),

Preferred conservative amino acids substitution groups are: valine-leucine-isoleucine, phenylalanine-tyrosine, lysine-arginine, alanine-valine, and asparagine-glutamine.

Accordingly, a variant or a fragment thereof according to the invention may comprise, within the same variant of the sequence or fragments thereof, or among different variants of the sequence or fragments thereof, at least one substitution, such as a plurality of substitutions introduced independently of one another.

It is clear from the above outline that the same variant or fragment thereof may comprise more than one conservative amino acid substitution from more than one group of conservative amino acids as defined herein above.

Aside from the twenty standard amino acids and two special amino acids, selenocysteine and pyrrolysine, there are a vast number of “nonstandard amino acids” which are not incorporated into protein in vivo. Examples of nonstandard amino acids include the sulfur-containing taurine and the neurotransmitters GABA and dopamine. Other examples are lanthionine, 2-Aminoisobutyric acid, and dehydroalanine. Further non standard amino are ornithine and citrulline.

Non-standard amino acids are usually formed through modifications to standard amino acids. For example, taurine can be formed by the decarboxylation of cysteine, while dopamine is synthesized from tyrosine and hydroxyproline is made by a posttranslational modification of proline (common in collagen). Examples of non-natural amino acids are those listed e.g. in 37 C.F.R. section 1.822(b)(4), all of which are incorporated herein by reference.

Both standard and non standard amino acid residues described herein can be in the “D” or “L” isomeric form.

It is contemplated that a functional equivalent according to the invention may comprise any amino acid including non-standard amino acids. In preferred embodiments a functional equivalent comprises only standard amino acids.

The standard and/or non-standard amino acids may be linked by peptide bonds or by non-peptide bonds. The term peptide also embraces post-translational modifications introduced by chemical or enzyme-catalyzed reactions, as are known in the art. Such post-translational modifications can be introduced prior to partitioning, if desired. Amino acids as specified herein will preferentially be in the L-stereoisomeric form. Amino acid analogs can be employed instead of the 20 naturally-occurring amino acids. Several such analogs are known, including fluorophenylalanine, norleucine, azetidine-2-carboxylic acid, S-aminoethyl cysteine, 4-methyl tryptophan and the like.

Suitably variants will be at least 60% identical, preferably at least 70% and accordingly, variants preferably have at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with the predetermined sequence of bovine alpha-lactalbumin.

Sequence identity can be calculated using a number of well-known algorithms and applying a number of different gap penalties. The sequence identity is calculated relative to full-length SEQ ID NO: 1 or SEQ ID NO: 2. In the alternative, it is calculated relative to SEQ ID NO: 1 or SEQ ID NO: 2, wherein the sequence encoding the signal peptide is not included. Without being bound by theory, the signal peptide is predicted to comprise amino acids 1 to 24 of SEQ ID NO: 1 and SEQ ID NO: 2. Any sequence alignment tool, such as but not limited to FASTA, BLAST, or LALIGN may be used for searching homologues and calculating sequence identity. Moreover, when appropriate any commonly known substitution matrix, such as but not limited to PAM, BLOSSUM or PSSM matrices may be applied with the search algorithm. For example, a PSSM (position specific scoring matrix) may be applied via the PSI-BLAST program. Moreover, sequence alignments may be performed using a range of penalties for gap opening and extension. For example, the BLAST algorithm may be used with a gap opening penalty in the range 5-12, and a gap extension penalty in the range 1-2.

A functional homologue within the scope of the present invention is a polypeptide that exhibits some sequence identity with bovine alpha-lactalbumin or with human alpha-lactalbumin as identified by SEQ ID NO: 1 or SEQ ID NO: 2, preferably they have a high sequence identity t SEQ ID NO: 1 or SEQ ID NO: 2, for example functional homologues may have a sequence sharing at least 70% sequence identity preferably functional homologues have at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with SEQ ID NO: 1 or SEQ ID NO: 2.

Functional equivalents may further comprise chemical modifications such as ubiquitination, labeling (e.g., with radionuclides, various enzymes, etc.), pegylation (derivatization with polyethylene glycol), or by insertion (or substitution by chemical synthesis) of amino acids (amino acids) such as ornithine, which do not normally occur in human proteins.

In addition to the peptidyl compounds described herein, sterically similar compounds may be formulated to mimic the key portions of the peptide structure and that such compounds may also be used in the same manner as the peptides of the invention. This may be achieved by techniques of modelling and chemical designing known to those of skill in the art. For example, esterification and other alkylations may be employed to modify the amino terminus of, e.g., a di-arginine peptide backbone, to mimic a tetra peptide structure. It will be understood that all such sterically similar constructs fall within the scope of the present invention.

Peptides with N-terminal alkylations and C-terminal esterifications are also encompassed within the present invention. Functional equivalents also comprise glycosylated and covalent or aggregative conjugates formed with the same molecules, including dimers or unrelated chemical moieties. Such functional equivalents are prepared by linkage of functionalities to groups which are found in fragment including at any one or both of the N- and C-termini, by means known in the art.

The term “fragment thereof” may refer to any portion of the given amino acid sequence. Fragments may comprise more than one portion from within the full-length protein, joined together. Suitable fragments may be deletion or addition mutants. The addition of at least one amino acid may be an addition of from preferably 2 to 250 amino acids, such as from 10 to 20 amino acids, for example from 20 to 30 amino acids, such as from 40 to 50 amino acids. Fragments may include small regions from the protein or combinations of these.

Suitable fragments may be deletion or addition mutants. The addition or deletion of at least one amino acid may be an addition or deletion of from preferably 2 to 250 amino acids, such as from 10 to 20 amino acids, for example from 20 to 30 amino acids, such as from 40 to 50 amino acids. The deletion and/or the addition may—independently of one another—be a deletion and/or an addition within a sequence and/or at the end of a sequence.

A functional homologue may be a deletion mutant of alpha-lactalbumin as identified by SEQ ID NO: 1 or SEQ ID NO: 2, sharing at least 70% and accordingly, a functional homologue preferably have at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity.

Deletion mutants suitably comprise at least 20 or 40 consecutive amino acid and more preferably at least 80 or 100 consecutive amino acids in length. Accordingly such a fragment may be a shorter sequence of the sequence as identified by SEQ ID NO: 1 or SEQ ID NO: 2 comprising at least 20 consecutive amino acids, for example at least 30 consecutive amino acids, such as at least 40 consecutive amino acids, for example at least 50 consecutive amino acids, such as at least 60 consecutive amino acids, for example at least 70 consecutive amino acids, such as at least 80 consecutive amino acids, for example at least 90 consecutive amino acids, such as at least 95 consecutive amino acids, such as at least 100 consecutive amino acids, such as at least 105 amino acids, for example at least 110 consecutive amino acids, such as at least 115 consecutive amino acids, for example at least 120 consecutive amino acids, wherein said deletion mutants preferably share at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with SEQ ID NO: 1 or SEQ ID NO:2.

It is preferred that functional homologues of alpha-lactalbumin comprises at the most 500, more preferably at the most 400, even more preferably at the most 300, yet more preferably at the most 200, such as at the most 175, for example at the most 160, such as at the most 150 amino acids, for example at the most 142 amino acids.

The term “fragment thereof” may refer to any portion of the given amino acid sequence. Fragments may comprise more than one portion from within the full-length protein, joined together. Portions will suitably comprise at least 5 and preferably at least 10 consecutive amino acids from the basic sequence. They may include small regions from the protein or combinations of these.

In an embodiment of the invention the alpha-lactalbumin fragment comprise one or more amino acid segments. The segments may be selected from the major alpha-helical domain containing amino acid 5-11, 23-34, 86-98, and the short alpha-helical segments; amino acid 18-20, 115-118, or from the beta-domain containing the triple-stranded anti-parallel sheet: amino acids 40-50 and the short 76-82 helix or the calcium binding domain 76-89 or any segments between these domains: amino acid 1-4, 12-17, 21-22, 35-39, 51-76, 82-84, 99-114 or 119-123. Preferably an alpha-lactalbumin fragment comprises at least two of the above mentioned segments, more preferably at least three of the indicated segments, more preferably four or most preferably five or all sixth mentioned segments.

The region which forms the interface between the alpha and beta domains is, in human a-lactalbumin, defined by amino acids 35-39 and 83-87 in the structure. Thus by parallel thereto suitable fragments of bovine alpha-lactalbumin will include these regions, and preferably the entire region from amino acid 35-87 of the native protein, for example from amino acid 20-100 of the native protein, for example from amino acid 10-110 of the native protein, for example from amino acid 5-115 of the native protein, for example from amino acid 1-123. This region of the molecule differs between the bovine and the human proteins, in that one of the three basic amino acids (R70) is changed to S70 in bovine alpha-lactalbumin, thus eliminating one potential coordinating side chain.

The deletion and/or the addition may—independently of one another—be a deletion and/or an addition within a sequence and/or at the end of a sequence.

The high affinity Ca²⁺ binding site is 100% conserved in alpha-lactalbumin from different species (Acharya K. R., et al., (1991) J Mol Bio 3 221, 5il-581), illustrating the importance of this function for the protein. It is co-ordinated by five different amino acids and two water molecules as described in the background section.

In a particular embodiment, a variant according to the invention is one in which the calcium binding site has been modified so that the affinity for calcium is reduced, or it is no longer functional. The calcium binding site in alpha-lactalbumin is coordinated by the residues K79, D82, D84, D87 and D88. Thus modification of these residues, by for example by removing one of more of the acidic residues, can reduce the affinity of the site for calcium, or eliminate the function completely and mutants of this type are an embodiment of the invention. In a specific embodiment, the aspartic acid residue at amino acid position 87 within the protein sequence is mutated to a non-acidic residue, and in particular a non-polar or uncharged polar side chain. In order to minimize the structural distortion in the mutant protein, D87 may also be replaced by an asparagine (N). Thus variants for use in the complexes of the invention may be D87A and D87N variants of a-lactalbumin, or fragments which include this mutation.

LAC appears to be active with and without calcium present. Two explanations for this are plausible. In the first and most likely scenario, LAC is formed by unfolding and binding of fatty acid (se below) with little disturbance of the alpha-helical domain. The Ca²⁺-binding site may then retain a similar conformation as in the absence of fatty acid and Ca²⁺ may be bound there to. A second possibility is that the Ca²⁺ site is disrupted and that the observed Ca²⁺ binding is explained by the generation of a new Ca²⁺ site in LAC. The head group of the fatty acid might potentially coordinate calcium together with amino acid residues.

It appears therefore that the Ca²⁺-binding site is not involved in the conversion of a-lactalbumin to an apoptosis-associated conformation, and that the structural changes associated with Ca²⁺ binding to LAC do not hinder the biological function. Thus in an alternative embodiment the Ca²⁺ binding site is preserved by the inclusion of amino acid segment 76-89 as described above.

### Alpha-Lactalbumin Complex

The alpha-lactalbumin composition according to the invention comprises an alpha-lactalbumin complex (LAC) comprising bovine or human alpha-lactalbumin of SEQ ID NO.1 or SEQ ID NO:2 or a functional equivalent there of and a lipid or a fatty acid.

In preferred embodiments of the present invention alpha-lactalbumin is complexed with a fatty acid. Fatty acids are carboxylic acids, which often have a long unbranched aliphatic chain. As the biosynthesis of fatty acids involves acetyl-CoA, in which the acetic unit contains two C-atoms, most natural fatty acids have an even number of C atoms ranging from 4 to 80 C atoms. The aliphatic chain of a fatty acid can be either saturated or unsaturated. Saturated fatty acids are saturated with hydrogen and thus have no double bonds. Unsaturated fatty acids can be either mono-unsaturated (or MUFAs), having one double bond or poly-unsaturated (PUFAs), having 2 or more double bonds. The fatty acids of the present invention may be a saturated fatty acid or an unsaturated fatty acid.

In preferred embodiments of the invention the fatty acid is selected from the group of C4 to C30, for example from C6 to C28, such as from C8 to C26, for example from C10 to C24, such as from C12 to C22, for example from C14 to C20, such as from C16 to C20, for example from the group of C16, C17, C18 and C20, such as from the group of C16, C18 and C20.

Fatty acids are often described using the number of C-atoms of the chain and the number, location and conformation of double bonds. Steric acid, for example, has a chain of 18 C-atoms and no double bonds and can be described as C18:0, oleic acid has a chain of 18 C-atoms and one double bond and can be described as C18:1, linoleic acid has a chain of 18 C-atoms and two double bonds and can be described as C18:2 and so forth.

The double bond is located on the xth carbon-carbon bond, counting from the carboxyl terminus. The Latin prefixes cis (on the side) or trans (across) describe the conformation of the double bonds by describing the orientation of the hydrogen atoms with respect to said double bond. Double bonds in the cis conformation are preferred. The position of the double bond is frequently indicated as the last number, following the integer indicating the number of double bonds. Thus, for example oleic acid having a 18 carbon chain with one double bond between carbon 9 and 10 may be described as C18:1:9cis and α-linolenic acid having a 18 carbon chain with three double bonds between carbon 9 and 10, 12 and 13 and 15 and 16, respectively, may be described as C18:3:9, 12, 15. Cis or trans may be indicated after the position of the double bond. If there is more than one double bond and they all are of the same conformation, then the term cis or trans may be indicated after indication of the position of all double bonds and thus relates to the conformation of all double bonds. Thus, for example Linoleic acid having a 18 carbon chain with 2 double bonds, which are both cis double bonds between carbons 9 and 10 and 12 and 13, respectively may be described as C18:2:9,12cis

In preferred embodiments of the present invention the fatty acid has in the range of 0 to 6 double bonds, for example in the range of 1 to 5 double bonds, such as the number of double bonds is selected from the group of 1, 2, 3 or 4 double bonds. In more preferred embodiments of the invention the fatty acid has 1 or 3 double bonds. In a most preferred embodiment of the invention the fatty acid has one double bond.

### Examples of Saturated Fatty Acids are:

Butyric (butanoic acid): CH3(CH2)2COOH or C4:0
Caproic (hexanoic acid): CH3(CH2)4COOH or C6:0
Caprylic (octanoic acid): CH3(CH2)6COOH or C8:0
Capric (decanoic acid): CH3(CH2)8COOH or C10:0
Lauric (dodecanoic acid): CH3(CH2)10COOH or C12:0
Myristic (tetradecanoic acid): CH3(CH2)12COOH or C14:0
Palmitic (hexadecanoic acid): CH3(CH2)14COOH or C16:0
Stearic (octadecanoic acid): CH3(CH2)16COOH or C18:0
Arachidic (eicosanoic acid): CH3(CH2)18COOH or C20:0
Behenic (docosanoic acid): CH3(CH2)20COOH or C22:0

Unsaturated fatty acids are preferred for the present invention.

Examples of Unsaturated Fatty Acids that May be Used in the Invention Include for Example:
Oleic acid: CH3(CH2)7CH═CH(CH2)7COOH or C18:1:9cis
Linoleic acid: CH3(CH2)4CH═CHCH2CH═CH(CH2)7COOH or C18:2:9,12cis

### Alpha-Linolenic Acid:

### CH3CH2CH═CHCH2CH═CHCH2CH═CH(CH2)7COOH or C18:3:9,12,15cis

### Arachidonic Acid:

### CH3(CH2)4CH═CHCH2CH═CHCH2CH═CHCH2CH═CH(CH2)3COOH or C20:4

Eicosapentaenoic acid or C20:5
Docosahexaenoic acid or C22:6
Erucic acid: CH3(CH2)7CH═CH(CH2)11COOH or C22:1
Vaccenic acid: C18:1:11cis
Palmitoleic acid: 16:1:9cis
Petroselinic acid: C18:1:6cis
Stearidonic acid: C18:4:6, 9, 12, 15cis

### Heptadecenoic Acid 17:1:10cis

Stearidonic acid 18:4:6,9,12,15cis

### Eicosenoic Acid 20:1:11cis

In an embodiment a mono-saturated acid is complexed with alpha-lactalbumin. More preferred are mono-saturated acids selected from the group of: C16:1:6cis and trans, C16:1:9cis and trans, C16:1:11 cis and trans, C18:1:6cis or trans, C18:1:9cis and trans, C18:1:11 cis or trans, C18:1:13cis or trans, C20:1:9 cis and trans, C20:1:11cis and trans, C20:1:13cis and trans.

In a preferred embodiment the mono-saturated acid complexed with alpha-lactalbumin is in the cis conformation such a fatty acid selected from the group of: C16:1:6cis, C16:1:9cis, C16:1:11cis, C18:1:6cis, C18:1:9cis, C18:1:11 cis, C18:1:13cis, C20:1:9cis, C20:1:11cis, C20:1:13cis.

In another preferred embodiment the fatty acid complexed with alpha-lactalbumin is an unsaturated fatty acid in the cis conformation, preferably selected from the group consisting of C18:1:9cis, C18:1:11cis, C18:1:6cis, C16:1:9cis, C18:3:6,9,12cis, C18:3:9,12,15cis, C18:2:9,12cis.

In another preferred embodiment the fatty acid complexed with alpha-lactalbumin is selected from the group consisting of C16 to C20 fatty acids comprising in the range of 1 to 5 cis double bonds. Thus, the fatty acid may for example be selected from the group consisting of Vaccenic Acid C18:1:11 cis, Linoleic Acid C18:2:9,12cis, Alpha Linolenic Acid C18:3:9,12,15, Palmitoleic Acid C16:1:9cis, Heptadecenoic Acid C17:1:10cis, Gamma Linolenic Acid C18:3:6,9,12cis, Stearidonic acid C18:4:6,9,12,15cis, Eicosenoic Acid C20:1:11 cis and Eicosapentaenoic Acid C20:5:5,8,11,14,17cis, such as from the group consisting of Vaccenic Acid C18:1:11cis, Linoleic Acid C18:2:9,12cis, Alpha Linolenic Acid C18:3:9,12,15

In a very preferred embodiment of the invention the fatty acid complexed with alpha-lactalbumin is an unsaturated C16 or C18 fatty acid, preferably a C18 fatty acid, wherein all double bonds are cis double bonds. In this embodiment the fatty acid may preferably comprise 1, for example 2, such as 3, for example 4 double bonds, wherein all double bonds are cis double bonds. Thus, the fatty acid may for example be selected from the group consisting of C18:1:9cis, C18:1:11 cis, C18:1:6cis, C16:1:9cis, C18:3:6,9,12cis, C18:3:9,12,15cis, C18:2:9,12cis and C18:4:6, 9, 12, 15cis, preferably selected from the group consisting of C18:1:9cis, C18:1:11 cis, C18:1:6cis, C18:3:6,9,12cis, C18:3:9,12,15cis, C18:2:9,12cis and C18:4:6, 9, 12, 15cis, for example selected from the group consisting of C18:1:9cis, C18:1:11 cis, C18:3:6,9,12cis, C18:3:9,12,15cis and C18:2:9,12cis.

Most preferred fatty acids are according to the invention C18:1:9cis and C18:1:11cis. C18:1:9cis is highly preferred for the complex of the invention.

In an alternative embodiment a polysaturated acid is complexed with alpha-lactalbumin. Preferably a polysaturated acid selected from the group of C18:2:9,12cis, C18:3:9,12,15cis, C18:3:6,9,12cis, and C20:4:5,8,11 15cis,

In one embodiment the fatty acid is an artificial fatty acid.

The fatty acid or lipid binding site in alpha-lactalbumin may be located in the groove between the α-helical and β-sheet domains, which becomes exposed in the apo-protein. The applicant believes that the cofactor such as oleic acid binds in the interface between the alpha and the beta domains, and that the bound cofactor acid locks this region of the molecule, while allowing the α-domain to maintain a native-like conformation.

This region of the molecule differs between the bovine and the human proteins, in that one of the three basic amino acids (R70) is changed to S70 in bovine α-lactalbumin, thus eliminating one potential coordinating side chain.

The active complex is preferably produced in local environments that favour the altered protein fold, and where fatty acid or lipid cofactors are available.

### Production of Alpha-Lactalbumin, Preferably LAC, Complex Composition

The alpha-lactalbumin composition may according to the invention be produced by any suitable methods. Natural sources of alpha-lactalbumin are milk from different mammalian species, preferably selected from the group of: equine, caprine, bovine and porcine, most preferably bovine. Alternatively alpha-lactalbumin may be produced recombinantly (see more details herein below in the section “recombinant production”) or obtained as a commercial product from several companies.

### Purification

Purification of proteins in general involves one or more steps of removal of or separation from contaminating nucleic acids, phages and/or viruses, other proteins and/or other biological macromolecules. The obtaining of LA from a composition comprising LA, such as milk or a culture medium or an extract of host cells (see herein below in the section “Recombinant production”) may comprise one or more protein isolation steps. Any suitable protein isolation step may be used with the present invention. The skilled person will in general readily be able to identify useful protein isolation steps for LA if such are required.

The protein isolation steps useful with the present invention may be commonly used methods for protein purification including for example chromatographic methods such as for example gas chromatography, liquid chromatography, ion exchange chromatography and/or affinity chromatography; filtration methods such as for example gel filtration and ultrafiltration; precipitation, such as ammonium sulphate precipitation and/or gradient separation such as sucrose gradient separation. Purification of LA may comprise one or more of the aforementioned methods in any combination.

The aforementioned methods are well known to the skilled person and may for example be performed as described in the “Protein Separation Handbook Collection” including the titles “Antibody Purification”, “The Recombinant Protein Handbook”, “Protein Purification”, “Ion Exchange Chromatography”, “Affinity Chromatography”, “Hydrophobic Interaction Chromatography”, “Gel Filtration”, “Reversed Phase Chromatography”, “Expanded Bed Adsorption” and “Chromatofocusing” prepared by Amersham Biosciences and available from GE.

In particular, purification of LA may for example comprise one or more centrifugation steps. Said centrifugation may be employed for example for defattening purposes and/or to remove cells/cellular debris or the like and/or to separate supernatant from precipitate

In particular, purification of LA may for example comprise one or more precipitation steps, for example precipitation using ammonium sulphate, for example at a concentration in 10 to 75%, preferably in the range of 30 to 60%, such as in the range of 40-45%. When precipitation is performed using an ammonium sulphate concentration of in the range of 40 to 45%, LA will generally be present in the supernatant.

Purification of LA may comprise one or more steps of filtration, for example filtration through a filter paper and/or filtration using another filter with a pore size of the range of 0.1 μm to 100 μm, for example in the range of 0.5 to 50 μm, such as in the range of 0.5 to 20 μm, such as in the range of 0.5-1 μm.

Purification of LA may comprise one or more chromatographic steps, for example any of the chromatographic methods mentioned above. In one preferred embodiment the method comprises a hydrophobic interaction chromatography.

### Recombinant Production

Functional equivalents of LA are preferably produced recombinantly. Wild type LA may in one preferred embodiment also be recombinantly produced. Useful recombinant production methods includes conventional methods known in the art, such as by expression of heterologous LA of functional homologues thereof in suitable host cells such as E. coli, S. cerevisiae or S. pombe or insect or mammalian cells suitable for production of recombinant proteins (see below). The skilled person will in general readily be able to identify useful recombinant techniques for the production of recombinant proteins in general and LA specifically.

In one embodiment LA is produced in a transgene plant or animal. By a transgenic plant or animal in this context is meant a plant or animal which has been genetically modified to contain and express a nucleic acid encoding human or bovine LA or functional homologous hereof.

In a preferred embodiment of the invention, LA or a functional homologue thereof is produced recombinantly by host cells.

Thus, in one aspect of the present invention, LA is produced by host cells comprising a first nucleic acid sequence encoding alpha-lactalbumin or a functional homologue thereof operably associated with a second nucleic acid capable of directing expression in said host cells. The second nucleic acid sequence may thus comprise or even consist of a promoter that will direct the expression of protein of interest in said cells. A skilled person will be readily capable of identifying useful second nucleic acid sequence for use in a given host cell.

The process of producing recombinant LA or a functional homologue thereof in general comprises the steps of:
providing a host cell
preparing a gene expression construct comprising a first nucleic acid encoding LA or a functional homologue thereof operably linked to a second nucleic acid capable of directing expression of said protein of interest in the host cell
transforming the host cell with the construct,
cultivating the host cell, thereby obtaining expression of LA or the functional homologue thereof.

The composition comprising LA may thus be an extract of said host cells or a composition purified from an extract of said host cells and/or from the culture medium.

The recombinant LA thus produced may be isolated by any conventional method for example by any of the protein purification methods described herein above. The skilled person will be able to identify a suitable protein isolation steps for purifying any protein of interest.

In one embodiment of the invention, the recombinantly produced LA or the functional homologue thereof is excreted by the host cells.

When the LA or the functional homologue thereof is excreted the process of producing a recombinant protein of interest may comprise the following steps
providing a host cell
preparing a gene expression construct comprising a first nucleic acid encoding LA or a functional homologue thereof operably linked to a second nucleic acid capable of directing expression of said LA or functional homologue thereof in said host cell
transforming said host cell with the construct,
cultivating the host cell in a culture medium, thereby obtaining expression of LA or the functional homologue thereof and secretion of the protein into the culture medium,
thereby obtaining culture medium comprising LA or a functional homologue thereof.

The composition comprising LA or a functional homologue thereof may thus in this embodiment of the invention be the culture medium or a composition prepared from the culture medium.

In another embodiment of the invention said composition is an extract prepared from animals, parts thereof or cells or an isolated fraction of such an extract.

In a preferred embodiment of the invention, LA is recombinantly produced in vitro in host cells and is isolated from cell lysate, cell extract or from tissue culture supernatant. In a more preferred embodiment LA is produced by host cells that are modified in such a way that they express the protein of interest. In an even more preferred embodiment of the invention said host cells are transformed to produce and excrete LA.

Thus in a preferred embodiment, the LA preparation is preferably a recombinant preparation, wherein the LA preparation is obtained by:
preparing a gene expression construct comprising a first nucleic acid encoding human or bovine alpha-lactalbumin peptide or a functional homologue thereof, operably linked to a second nucleic acid capable of directing expression in a host cell,
transforming a host cell culture with the construct,
cultivating the host cell culture in a culture medium, thereby obtaining expression and secretion of the polypeptide into the culture medium
obtaining a composition comprising a variety of alpha-lactalbumin molecules and nucleic acids

In one embodiment the LA preparation is preferably a recombinant preparation, wherein the LA preparation is obtained by:
preparing a gene expression construct comprising a first nucleic acid encoding human or bovine alpha-lactalbumin peptide or a functional homologue thereof, operably linked to a second nucleic acid capable of directing expression in a host cell,
transforming a host cell culture with the construct,
cultivating the host cell culture either in vitro or in the form of a transgenic plant or animal thereby obtaining expression of LA
obtaining a composition comprising a plurality of LA molecules and nucleic acids

According to the invention, the nucleic acid encoding alpha-lactalbumin may be derived from the human or bovine alpha-lactalbumin gene or from alpha-lactalbumin genes of other animal species as defined herein above.

In a preferred embodiment the gene expression construct is suitable for expression in mammalian cell lines or transgenic plants or animals. In one embodiment the host cell culture is cultured in a transgene animal. By a transgenic plant or animal in this context is meant a plant or animal which has been genetically modified to contain and express a nucleic acid encoding human or bovine alpha-lactalbumin or a functional homologue thereof as defined herein above

In a preferred embodiment the gene expression construct of the present invention comprises a viral based vector, such as a DNA viral based vector, a RNA viral based vector, or a chimeric viral based vector. Examples of DNA viruses are cytomegalo virus, Herpex Simplex, Epstein-Barr virus, Simian virus 40, Bovine papillomavirus, Adeno-associated virus, Adenovirus, Vaccinia virus, and Baculo virus. However, the gene expression construct may for example only comprise a plasmid based vector.

In one aspect the invention provides an expression construct encoding human or bovine alpha-lactalbumin or functional homologues thereof, featured by comprising one or more intron sequences from the human or bovine human or bovine alpha-lactalbumin gene including functional derivatives hereof. Additionally, it may contain a promoter region derived from a viral gene or an eukaryotic gene, including mammalian and insect genes.

The promoter region is preferably selected to be different from the native human or bovine human or bovine alpha-lactalbumin promoter, and preferably in order to optimize the yield of human or bovine alpha-lactalbumin, the promoter region is selected to function most optimally with the vector and host cells in question.

In a preferred embodiment the promoter region is selected from a group comprising Rous sarcoma virus long terminal repeat promoter, and cytomegalovirus immediate-early promoter, and elongation factor-1 alpha promoter.

In another embodiment the promoter region is derived from a gene of a microorganism, such as other viruses, yeasts and bacteria.

In order to obtain a greater yield of recombinant LA or functional homologue thereof, the promoter region may comprise enhancer elements, such as the QBI SP163 element of the 5′ end untranslated region of the mouse vascular endothelian growth factor gene

One process for producing recombinant LA according to the invention is characterised in that the host cell culture is may be eukaryotic, and for example a mammalian cell culture or a yeast cell culture.

Useful mammalian cells may for example be human embryonal kidney cells (HEK cells), such as the cell lines deposited at the American Type Culture Collection with the numbers CRL-1573 and CRL-10852, chick embryo fibroblast, hamster ovary cells, baby hamster kidney cells, human cervical carcinoma cells, human melanoma cells, human kidney cells, human umbilical vascular endothelium cells, human brain endothelium cells, human oral cavity tumor cells, monkey kidney cells, mouse fibroblast, mouse kidney cells, mouse connective tissue cells, mouse oligodendritic cells, mouse macrophage, mouse fibroblast, mouse neuroblastoma cells, mouse pre-B cell, mouse B lymphoma cells, mouse plasmacytoma cells, mouse teratocacinoma cells, rat astrocytoma cells, rat mammary epithelium cells, COS, CHO, BHK, 293, VERO, HeLa, MDCK, W138, and NIH 3T3 cells.

It is however preferred that the host cells are either prokaryotic cells or yeast cells. Prokaryotic cells may for example be E. coli. Yeast cells may for example be Saccharomyces, Pichia or Hansenula.

When recombinantly produced alpha-lactalbumin is used with the present invention it is preferred that said recombinantly produced alpha-lactalbumin has a size distribution profile that is similar to naturally occurring alpha-lactalbumin.

The aforementioned methods are well known to the skilled person and may for example be performed as described in the Current Protocols in Molecular Biology, 2001, by John Wiley and Sons, Inc. edited by Frederick M. Ausubel et al.

Recombinantly produced LA may for example be purified as described herein above in the section “Purification of Alpha-lactalbumin” and recombinantly produced LA may be used for preparing LAC, for example as described herein below.

### Method of Producing Alpha-Lactalbumin Complex

LAC according to the present invention is an active complex of alpha-lactalbumin and a fatty acid or lipid. Functional assays known to a skilled person can be used to verify the functional activity of alpha-lactalbumin in complex with a fatty acid or a lipid. Functional assays for evaluating alpha-lactalbumin function known to persons skilled in the art include, but are not limited to, assays described herein above and in examples 6 and 7, such as the cell killing assay or the histone assay.

A preferred method of production of the alpha-lactalbumin complex, preferably LAC, composition starting from cow milk may comprise steps of centrifugation, precipitations, filtering and hydrophobic interaction chromatography. A preferred method of production of alpha-lactalbumin from milk is described in example 1. The purification of alpha-lactalbumin is followed by conversion of alpha-lactalbumin to alpha-lactalbumin complex, preferably LAC. This conversion may be performed by a series of steps including release of Ca²⁺ ion from alpha-lactalbumin. Alpha-lactalbumin is then allowed to bind the lipid cofactor, for example, on an ion exchange matrix. Third, the active complex may be isolated by e.g. elution using high salt concentration (see example 1).

### Release of Calcium

Release of calcium may be obtained by any suitable method known to the skilled person.

In one embodiment release of calcium may be achieved by contacting LA with a calcium chelating agent. The calcium chelating agent may be selected from the group of calcium chelators comprising, but not limited to 1,2-Bis(2-aminophenoxy)-ethane-N,N,N′,N′-tetraacetic acid (BAPTA) or Ethylene glycol-bis(aminoethylether)-N,N,N′,N′-tetraacetic (EGTA) or Ethylene diamine tetraacetic acid (EDTA). In a very preferred embodiment of the invention the calcium chelator is Ethylene diamine tetraacetic acid (EDTA).

In one preferred embodiment the calcium chelating agent is ethylene diamine tetraacetic acid.

In a special embodiment of the invention, release of calcium is obtained by using a functional homologue of alpha-lactalbumin, wherein the calcium binding site has been modified in a manner that reduces the ability of said functional homologue of alpha lactalbumin to bind calcium. In particular, the amino acids of the calcium-binding site (K79, D82, D84, D87 and D88) may be modified so that the affinity for calcium is reduced, or it is no longer functional. In this special embodiment the step involving the release of calcium from LA is obsolete and the conversion of LA to LAC comprises of the binding of a fatty acid or a lipid to LA with either simultaneous or subsequent exposure to an anion exchange medium.

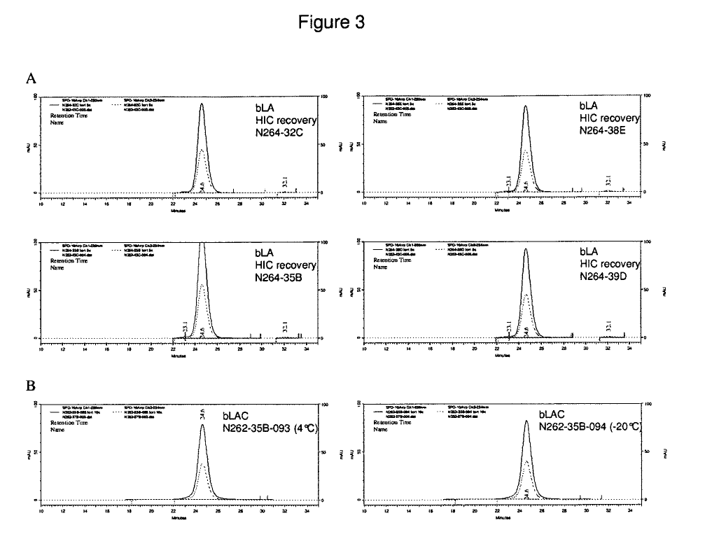

The alpha-lactalbumin composition may further be analysed using poly acryl gel electrophoresis (PAGE), immunoblotting (western blotting) and Size Exclusion Chromatography (SEC), MALDI-MS or any other methods whereby the content of the alpha-lactalbumin composition may be analysed. Analysis of an alpha-lactalbumin composition using poly acryl gel electrophoresis (PAGE), immuno-blotting (western blotting) and Size Exclusion Chromatography (SEC), is shown in FIGS. 3 and 4.

Following, the purified alpha-lactalbumin complex may be filtered, concentrated and buffer changed to have a stable solution of the alpha-lactalbumin complex in a high concentration.

In preferred embodiment the composition is a saline composition of 0.01 to 90% such as 0.1 to 80% NaCl, for example 0.2 to 70% NaCl, such as 0.3 to 60% NaCl, for example 0.4 to 50% NaCl, such as 0.5 to 40% NaCl, for example 0.6 to 30% NaCl, such as 0.7 to 20% NaCl, such as 0.8 to 10% NaCl, for example 0.85 to 5% NaCl, such as around 0.9% NaCl. In a preferred embodiment the composition is a 0.9% NaCl solution.

The alpha-lactalbumin complex, preferably LAC, composition according to the invention preferably has a concentration of more than 1 mg/ml, such as more than 2 mg/ml, preferably more than 5 mg/ml, such as more than 6 mg/ml or 7 mg/ml such as more than 8 mg/ml more preferably approximately 9 mg/ml. In another embodiment the alpha-lactalbumin complex, preferably LAC, composition according to the invention preferably has a concentration of approximately 7 mg/ml.

The applicant have found, with out being bound by the theory, that the ratio of monomeric/multimeric alpha-lactalbumin in the alpha-lactalbumin complex, preferably LAC, composition is controlled by the ratio of monomeric/multimeric alpha-lactalbumin subjected to the conversion procedure. Thus the ratio of monomeric/-multimeric alpha-lactalbumin obtained from the initial purification procedure of alpha-lactalbumin is important. In example 1, the output of hydrophobic interaction chromatography (HIC) is as seen in FIG. 3A is predominantly monomeric, leading to a monomeric alpha-lactalbumin complex composition (FIG. 3B), whereas a purification procedure giving rise to a mixed monomeric/multimeric alpha-lactalbumin composition results in a mixed monomeric/multimeric alpha-lactalbumin complex composition (data not shown). In the method applied in example 1, alpha-lactalbumin is obtained following HIC with a load of 6 mg/ml gel (90 mg/cm²), whereas mixed monomeric/multimeric alpha-lactalbumin composition have been obtained following HIC with a load of 2 mg/ml gel (32 mg/cm²). Production of monomeric alpha-lactalbumin complex may be favoured by a high concentration of the starting product, whereby the intermediated HIC purified alpha-lactalbumin is obtained in a monomeric form suitable for conversion resulting in an alpha-lactalbumin complex in the monomeric form.

An aspect of the invention accordingly relates to a method of producing a composition according to the invention comprising more than 95% (weight %) monomeric alpha-lactalbumin complex, preferably LAC in comparison to the content of multimeric or oligomeric LAC.

An embodiment relates to a method of producing a composition comprising monomeric LAC, which is a monomeric complex of alpha-lactalbumin and a fatty acid or a lipid, said alpha-lactalbumin being bovine or human alpha-lactalbumin of SEQ ID NO:1 or SEQ ID NO:2 or a functional equivalent thereof, wherein the composition comprises at least 50% by weight of monomeric alpha-lactalbumin comprising the steps of:
b. obtaining an alpha-lactalbumin composition comprising at least 95% by weight of monomeric alpha-lactalbumin,
c. conversion of said alpha-lactalbumin to LAC
   i. by release of calcium from said alpha-lactalbumin and
   ii. binding of fatty acid or lipid to said alpha-lactalbumin, and
d. purification of LAC.

The steps ci and cii may be performed sequentially in any order, or simultaneously.

The alpha-lactalbumin composition comprising at least 95% by weight of monomeric alpha-lactalbumin may preferably be obtained by hydrophobic interaction chromatography, wherein the column is loaded with more than 2 mg/ml. preferably more than 4 mg/ml, such as at least 6 mg/ml. The load may alternatively be measured in mg/cm², whereby the preferred load is over 40 mg/cm², such as more than 50 or preferably more than 60 mg/cm². Even further preferred is a load of more than 70 mg/cm² or more than 80 mg/cm².

In some embodiments the methods used with the present invention for production of alpha-lactalbumin complex, preferably LAC involves conversion of alpha-lactalbumin to alpha-lactalbumin complex by chromatography, for example as described in Examples 3 and 4 in Danish Patent application PA 2007 00693. In these embodiments a higher load of alpha-lactalbumin and a high yield of alpha-lactalbumin complex, preferably LAC can be achieved.

Thus, for production of LAC the column for conversion may be loaded with more than 20 mg alpha-lactalbumin/cm² ion exchange medium, such as at least 30 mg/cm², for example more than 40 mg/cm², such as at least 50 mg/cm², for example more than 60 mg/cm², such as at least 70 mg/cm², for example more than 80 mg/cm², such as at least 90 mg alpha-lactabumin/cm² ion exchange medium. In an embodiment of the present invention the yield of lactalbumin complex with aforementioned load is at least 50%, such as at least 55%, for example more than 60%, such as at least 65%, for example more than 70%, such as at least 75%, for example more than 80%. Thus, in one preferred embodiment the yield is at least 60%, preferably at least 70%, for example at least 75, such as at least 80%, when the load is 30 mg alpha-lactalbumin/cm² ion exchange medium. It is also preferred that the yield is at least 60%, preferably at least 70%, for example at least 75, such as at least 80%, for example at least 90%, when the load is 42 mg alpha-lactalbumin/cm² ion exchange medium. In another embodiment it is preferred that the yield is at least 20%, preferably at least 30%, such as at least 60%, preferably at least 70%, for example at least 75, such as at least 80%, when the load is 90 mg alpha-lactalbumin/cm² ion exchange medium.

It is furthermore preferred that the composition comprises only very little—if any—contaminating biomacromolecules such as proteins. Thus it is preferred that at least 50%, such as at least 60%, for example at least 70%, such as at least 80%, for example at least 90% by weight of the proteins of the composition is LAC.

### Cytotoxicity

As described in the background section multimeric alpha-lactalbumin (MAL) or HAMLET have been demonstrated to poses selective cytotoxic activities towards cancer cells and immature cells besides its effect on bacterial and viral infections. Formation of the active complex has been shown to be dependent on the cofactor oleic acid and stimulated by depletion of ca²⁺ from the environment.

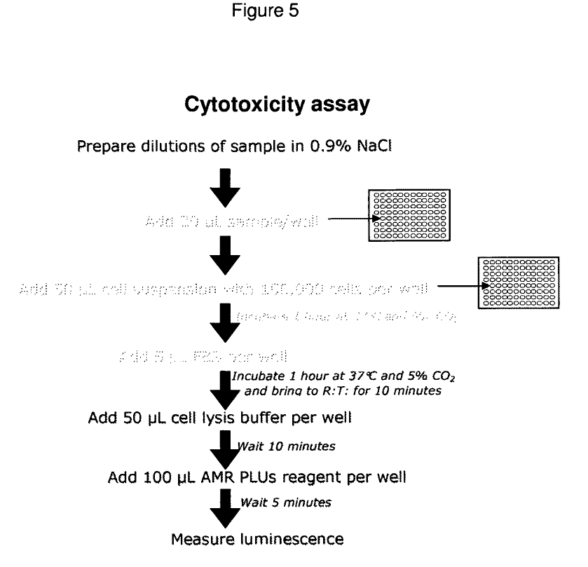

The applicant herein describes the production of monomeric alpha-lactalbumin complex, preferably LAC with similar cytotoxic activity towards cancer cells. The cytotoxicity of the monomeric alpha-lactalbumin complex, preferably LAC composition may be evaluated using any suitable cytotoxicity assay, which is well known in the art. An example of such an assay, namely the ViaLight assay is described in example 2. An overview of the procedure is outlined in FIG. 5.

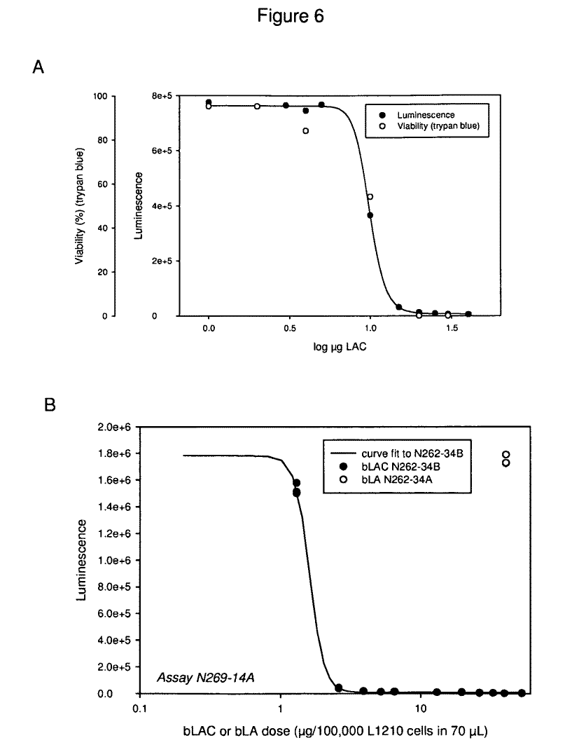

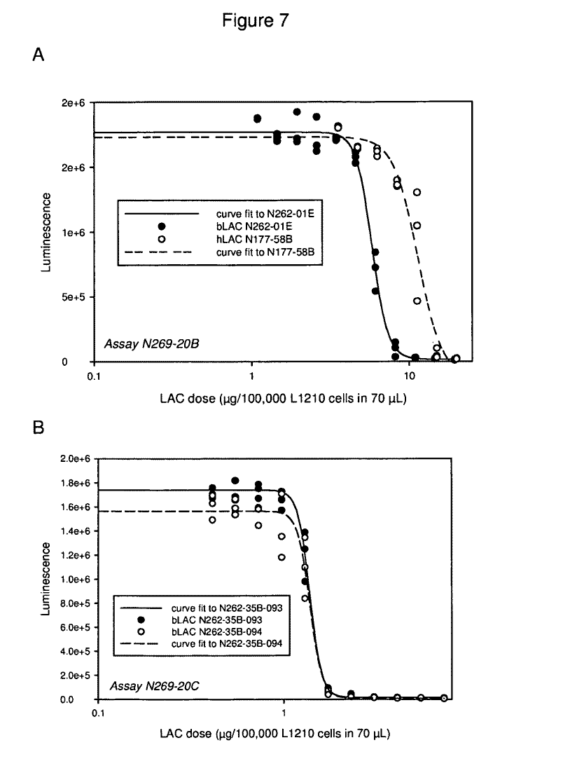

The dose of alpha-lactalbumin capable of killing 50% of a given cell population is calculated base on the measured luminescence data. The potency of the alpha-lactalbumin composition is reflected by the LD50 dose, where a low LD50 dose is characteristic for a composition with high potency, i.e. a composition highly effective in killing cancer cells. In this situation a cancer cell line L1210 is used, although it is clear that several different cell lines are suitable for the purpose. Results from such analysis are depicted in FIGS. 6 and 7 and in a table format in example 2, table 1.

In an embodiment the cytotoxic activity measured as LD50 of the alpha-lactalbumin complex, preferably LAC composition is less than 15 μg/100.000 cells in 70 μl. In a preferred embodiment the LD50 is less than 10 μg/100.000 cells in 70 μl. The alpha-lactalbumin complex, preferably LAC composition according to the invention more preferable has a cytotoxic activity measured as LD50 of less than 5 μg/100.000 cells. Most preferred is a composition wherein the cytotoxic activity measured as LD50 is 1-5 μg/100.000 cells in 70 μl.

The LD50 may be calculated as the concentration of the compound such as the amount of alpha-lactalbumin complex, preferably LAC composition required to kill 50% of a cell population in a predetermined volume. The results shown in table 1 example 2 shows that the composition according to the invention preferably have a LD50 concentration of less than 0.1 mg/ml, when measured on a cell population of 100,000 cells in 70 μl such as a LD50 of less than 0.9 mg/ml when measured on a cell population of 100.000 cells in 70 μl. In particular preferred embodiment the LD50 concentration of less than 0.08 when measured on a cell population of 100,000 cells in 70 μl, more preferred less than 0.06 most preferred less than 0.04 mg/ml when measured on a cell population of 100,000 cells in 70 μl.

### Pharmaceutical Formulation

Pharmaceutical compositions containing a composition of the present invention may be prepared by conventional techniques, e.g. as described in Remington: The Science and Practice of Pharmacy 1995, edited by E. W. Martin, Mack Publishing Company, 19th edition, Easton, Pa. The compositions may appear in conventional forms, for example capsules, tablets, aerosols, solutions, suspensions or topical applications.

The terms “medicament” and “pharmaceutical compostions” are used interchangeably herein.

The present invention provides pharmaceutical compositions comprising alpha-lactalbumin complex, preferably LAC.

In one aspect the present invention relates to a pharmaceutical composition. The pharmaceutical composition may be formulated in a number of different manners, depending on the purpose for the particular pharmaceutical composition.

For example the pharmaceutical composition may be formulated in a manner so it is useful for a particular administration form. Preferred administration forms are described herein below.

In one embodiment the pharmaceutical composition is formulated so it is a liquid. For example the composition may be a protein solution or the composition may be a protein suspension. Said liquid may be suitable for parenteral administration, for example for injection or infusion.

The liquid may be any useful liquid, however it is frequently preferred that the liquid is an aqueous liquid. For many purposes, in particular when the liquid should be used for parenteral administration, it is furthermore preferred that the liquid is sterile. Sterility may be conferred by any conventional method, for example filtration, irradiation or heating. Furthermore, it is preferred that the liquid has been subjected to a virus reduction step, in particular if the liquid is formulated for parenteral administration.

Virus reduction may for example be performed by nanofiltration or virus filtering over a suitable filter, such as a Planova filter consisting of several layers. The Planova filter may be any suitable size for example 75N, 35N, 20N or 15N or filters of different size may be used, for example Planova 20N. Virus reduction may also comprise a step of prefiltering with another filter, for example using a filter with a pore size of the range of 0.01 to 1 μm, such as in the range of 0.05 to 0.5 μm, for example around 0.1 μm. Virus reductions may also include an acidic treatment step.

The pharmaceutical composition may be packaged in single dosage units, which may be more convenient for the user. Hence, pharmaceutical compositions for bolus injections may be packages in dosage units of for example at the most 10 ml, preferably at the most 8 ml, more preferably at the most 6 ml, such as at the most 5 ml, for example at the most 4 ml, such as at the most 3 ml, for example around 2.2 ml.

The pharmaceutical composition may be packaged in any suitable container. In one example a single dosage of the pharmaceutical composition may be packaged in injection syringes or in a container useful for infusion.

In another embodiment of the present invention the pharmaceutical composition is a dry composition. The dry composition may be used as such, but for most purposes the composition is a dry composition for storage only. Prior to use the dry composition may be dissolved or suspended in a suitable liquid composition, for example sterile water.

The pharmaceutical composition according to the present invention may also comprise a first nucleic acid sequence encoding LAC, such as any of the LAC mentioned herein above. Said first nucleic acid sequence is preferably operably associated with a second nucleic acid sequence directing expression of the first nucleic acid in the individual to be treated with the pharmaceutical composition, more preferably in the cells of said individual, which are diseased. Thus it is preferred that the second nucleic acid sequence is capable of directing expression of the first nucleic acid sequence in a human being. In embodiments of the invention wherein the clinical condition is cancer, it is preferred that the second nucleic acid sequence is capable of directing expression of the first nucleic acid sequence in cancer cells, such as malignant cells. It is furthermore preferred that the first and the second nucleic acid sequences are included in a suitable vector.

It is also comprised within the invention that the pharmaceutical composition may be applied topically to the site of the site, for example in the form of a lotion, a crème, an ointment, a spray, such as an aerosol spray or a nasal spray, rectal or vaginal suppositories, drops, such as eye drops or nasal drops, a patch, an occlusive dressing or the like.

### Pharmaceutically Acceptable Additives

The pharmaceutical compositions containing alpha-lactalbumin complex, preferably LAC may be prepared by any conventional technique, e.g. as described in Remington: The Science and Practice of Pharmacy 1995, edited by E. W. Martin, Mack Publishing Company, 19th edition, Easton, Pa.

The terms “medicament” and “pharmaceutical compostions” are used interchangeably herein.

The pharmaceutically acceptable additives may be any conventionally used pharmaceutically acceptable additive, which should be selected according to the specific formulation, intended administration route etc. For example the pharmaceutically acceptable additives may be any of the additives mentioned in Nema et al, 1997. Furthermore, the pharmaceutically acceptable additive may be any accepted additive from FDA's “inactive ingredients list”, which for example is available on the internet address http://www.fda.gov/cder/drug/iig/default.htm.

In some embodiments of the present invention it is desirable that the pharmaceutical composition comprises an isotonic agent. In particular when the pharmaceutical composition is prepared for administration by injection or infusion it is often desirable that an isotonic agent is added.

Accordingly, the composition may comprise at least one pharmaceutically acceptable additive which is an isotonic agent.

The pharmaceutical composition may be isotonic, hypotonic or hypertonic. However it is often preferred that a pharmaceutical composition for infusion or injection is essentially isotonic, when it is administrated. Hence, for storage the pharmaceutical composition may preferably be isotonic or hypertonic. If the pharmaceutical composition is hypertonic for storage, it may be diluted to become an isotonic solution prior to administration.

The isotonic agent may be an ionic isotonic agent such as a salt or a non-ionic isotonic agent such as a carbohydrate.

Examples of ionic isotonic agents include but are not limited to NaCl, CaCl₂, KCl and MgCl₂. Examples of non-ionic isotonic agents include but are not limited to mannitol and glycerol.

However, in other embodiments of the invention the pharmaceutical composition may comprise no buffer at all or only micromolar amounts of buffer.

In a preferred embodiment the buffer is TRIS. TRIS buffer is known under various other names for example tromethamine including tromethamine USP, THAM, Trizma, Trisamine, Tris amino and trometamol. The designation TRIS covers all the aforementioned designations.

The buffer may furthermore for example be selected from USP compatible buffers for parenteral use, in particular, when the pharmaceutical formulation is for parenteral use. For example the buffer may be selected from the group consisting of monobasic acids such as acetic, benzoic, gluconic, glyceric and lactic, dibasic acids such as aconitic, adipic, ascorbic, carbonic, glutamic, malic, succinic and tartaric, polybasic acids such as citric and phosphoric and bases such as ammonia, diethanolamine, glycine, triethanolamine, and TRIS.

The pharmaceutical compositions may comprise at least one pharmaceutically acceptable additive which is a stabiliser.

For example the stabiliser may be selected from the group consisting of poloxamers, Tween-20, Tween-40, Tween-60, Tween-80, Brij, metal ions, amino acids, polyethylene glycol, Triton, EDTA, ascorbic acid, Triton X-100, NP40 or CHAPS.

The pharmaceutical composition according to the invention may also comprise one or more cryoprotectant agents. In particular, when the composition comprises freeze-dried protein or the composition should be stored frozen, it may be desirable to add a cryoprotecting agent to the pharmaceutical composition.

The cryoprotectant agent may be any useful cryoprotectant agent, for example the cryoprotectant agent may be selected from the group consisting of dextran, glycerin, polyethylenglycol, sucrose, trehalose and mannitol.

Accordingly, the pharmaceutically acceptable additives may comprise one or more selected from the group consisting of isotonic salt, hypertonic salt, buffer and stabilisers. Furthermore, the pharmaceutically acceptable additives may comprise one or more selected from the group consisting of isotonic agents, buffer, stabilisers and cryoprotectant agents. For example, the pharmaceutically acceptable additives comprise glucosemonohydrate, glycine, NaCl and polyethyleneglycol 3350.

### Formulations

Whilst it is possible for the composition of the present invention to be administered as the raw composition, it is preferred to present it in the form of a pharmaceutical formulation. Accordingly, the present invention further provides a pharmaceutical formulation, for medicinal application, which comprises a composition of the present invention or a pharmaceutically acceptable salt thereof, as herein defined, and a pharmaceutically acceptable carrier therefore.

### Oral Administration

The compositions of the present invention may be formulated in a wide variety of oral administration dosage forms. The pharmaceutical compositions and dosage forms may comprise the compositions of the invention or its pharmaceutically acceptable salt or a crystal form thereof as the active component. The pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, wetting agents, tablet disintegrating agents, or an encapsulating material.

Preferably, the composition will be about 0.5% to 75% by weight of a composition or compositions of the invention, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like.

In powders, the carrier is a finely divided solid which is a mixture with the finely divided active component. In tablets, the active component is mixed with the carrier having the necessary binding capacity in suitable proportions and compacted in the shape and size desired. The powders and tablets preferably contain from 1 to about 70% t of the active composition. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active composition with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration. Multiple-unit-dosage granules can be prepared as well. Tablets and granules of the above cores can be coated with concentrated solutions of sugar, etc. The cores can also be coated with polymers which change the dissolution rate in the gastrointestinal tract, such as anionic polymers having a pka of above 5.5. Such polymers are hydroxypropylmethyl cellulose phthalate, cellulose acetate phthalate, and polymers sold under the trade mark Eudragit S100 and L100. In preparation of gelatine capsules these can be soft or hard. In the former case the active compound is mixed with oil, and in the latter case the multiple-unit-dosage granules are filled therein.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. The resulting solution may then be clarified by filtration, transferred to a suitable container which is then sealed and sterilized by autoclaving or maintaining at 98-100 degree C. for half an hour. Alternatively, the solution may be sterilized by filtration and transferred to the container aseptically. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilising agents, and the like.

Other forms suitable for oral administration include liquid form preparations including emulsions, syrups, elixirs, aqueous solutions, aqueous suspensions, toothpaste, gel dentrifrice, chewing gum, or solid form preparations which are intended to be converted shortly before use to liquid form preparations. Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavours, stabilizing and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavours, stabilizers, buffers, artificial and natural sweeteners, dispersants, thickeners, solubilising agents, and the like.

### Parenteral Administration

The compositions of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-.beta.-aminopropionates, and 2-alkyl-imidazoline quaternary ammonium salts, and (e) mixtures thereof.

The parenteral formulations typically will contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

### Topical Administration

The compositions of the invention can also be delivered topically. Regions for topical administration include the skin surface and also mucous membrane tissues of the vagina, rectum, nose, mouth, and throat. Compositions for topical administration via the skin and mucous membranes should not give rise to signs of irritation, such as swelling or redness.

The topical composition may include a pharmaceutically acceptable carrier adapted for topical administration. Thus, the composition may take the form of a suspension, solution, ointment, lotion, sexual lubricant, cream, foam, aerosol, spray, suppository, implant, inhalant, tablet, capsule, dry powder, syrup, balm or lozenge, for example. Methods for preparing such compositions are well known in the pharmaceutical industry.

The compositions of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Creams, ointments or pastes according to the present invention are semi-solid formutations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

### Transdermal Delivery

The pharmaceutical agent-chemical modifier complexes described herein can be administered transdermally. Transdermal administration typically involves the delivery of a pharmaceutical agent for percutaneous passage of the drug into the systemic circulation of the patient. The skin sites include anatomic regions for trans-dermally administering the drug and include the forearm, abdomen, chest, back, buttock, mastoidal area, and the like.

Transdermal delivery is accomplished by exposing a source of the complex to a patient's skin for an extended period of time. Transdermal patches have the added advantage of providing controlled delivery of a pharmaceutical agent-chemical modifier complex to the body. See Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Such dosage forms can be made by dissolving, dispersing, or otherwise incorporating the pharmaceutical agent-chemical modifier complex in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

### Passive Transdermal Drug Delivery

A variety of types of transdermal patches will find use in the methods described herein. For example, a simple adhesive patch can be prepared from a backing material and an acrylate adhesive. The pharmaceutical agent-chemical modifier complex and any enhancer are formulated into the adhesive casting solution and allowed to mix thoroughly. The solution is cast directly onto the backing material and the casting solvent is evaporated in an oven, leaving an adhesive film. The release liner can be attached to complete the system.

Alternatively, a polyurethane matrix patch can be employed to deliver the pharmaceutical agent-chemical modifier complex. The layers of this patch comprise a backing, a polyurethane drug/enhancer matrix, a membrane, an adhesive, and a release liner. The polyurethane matrix is prepared using a room temperature curing polyurethane prepolymer. Addition of water, alcohol, and complex to the prepolymer results in the formation of a tacky firm elastomer that can be directly cast only the backing material.

A further embodiment of this invention will utilize a hydrogel matrix patch. Typically, the hydrogel matrix will comprise alcohol, water, drug, and several hydrophilic polymers. This hydrogel matrix can be incorporated into a transdermal patch between the backing and the adhesive layer.

The liquid reservoir patch will also find use in the methods described herein. This patch comprises an impermeable or semipermeable, heat sealable backing material, a heat sealable membrane, an acrylate based pressure sensitive skin adhesive, and a siliconized release liner. The backing is heat sealed to the membrane to form a reservoir which can then be filled with a solution of the complex, enhancers, gelling agent, and other excipients.

Foam matrix patches are similar in design and components to the liquid reservoir system, except that the gelled pharmaceutical agent-chemical modifier solution is constrained in a thin foam layer, typically a polyurethane. This foam layer is situated between the backing and the membrane which have been heat sealed at the periphery of the patch.

For passive delivery systems, the rate of release is typically controlled by a membrane placed between the reservoir and the skin, by diffusion from a monolithic device, or by the skin itself serving as a rate-controlling barrier in the delivery system. See U.S. Pat. Nos. 4,816,258; 4,927,408; 4,904,475; 4,588,580, 4,788,062; and the like. The rate of drug delivery will be dependent, in part, upon the nature of the membrane. For example, the rate of drug delivery across membranes within the body is generally higher than across dermal barriers. The rate at which the complex is delivered from the device to the membrane is most advantageously controlled by the use of rate-limiting membranes which are placed between the reservoir and the skin. Assuming that the skin is sufficiently permeable to the complex (i.e., absorption through the skin is greater than the rate of passage through the membrane), the membrane will serve to control the dosage rate experienced by the patient.

Suitable permeable membrane materials may be selected based on the desired degree of permeability, the nature of the complex, and the mechanical considerations related to constructing the device. Exemplary permeable membrane materials include a wide variety of natural and synthetic polymers, such as polydimethylsiloxanes (silicone rubbers), ethylenevinylacetate copolymer (EVA), polyurethanes, polyurethane-polyether copolymers, polyethylenes, polyamides, polyvinylchlorides (PVC), polypropylenes, polycarbonates, polytetrafluoroethylenes (PTFE), cellulosic materials, e.g., cellulose triacetate and cellulose nitrate/acetate, and hydrogels, e.g., 2-hydroxyethylmethacrylate (HEMA).

Other items may be contained in the device, such as other conventional components of therapeutic products, depending upon the desired device characteristics. For example, the compositions according to this invention may also include one or more preservatives or bacteriostatic agents, e.g., methyl hydroxybenzoate, propyl hydroxybenzoate, chlorocresol, benzalkonium chlorides, and the like. These pharmaceutical compositions also can contain other active ingredients such as antimicrobial agents, particularly antibiotics, anesthetics, analgesics, and antipruritic agents.

### Administration as Suppositories

The compositions of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The active composition may be formulated into a suppository comprising, for example, about 0.5% to about 50% of a composition of the invention, disposed in a polyethylene glycol (PEG) carrier (e.g., PEG 1000 [96%] and PEG 4000 [4%].

The compositions of the present invention may be formulated for vaginal administration. Pessaries, tampons, creams, gels, pastes, foams or sprays containing in addition to the active ingredient such carriers as are known in the art to be appropriate.

### Respiratory Tract Administration

The compositions of the present invention may be formulated for nasal administration. The solutions or suspensions are applied directly to the nasal cavity by conventional means, for example with a dropper, pipette or spray. The formulations may be provided in a single or multidose form. In the latter case of a dropper or pipette this may be achieved by the patient administering an appropriate, predetermined volume of the solution or suspension. In the case of a spray this may be achieved for example by means of a metering atomizing spray pump.

The compositions of the present invention may be formulated for aerosol administration, particularly to the respiratory tract and including intranasal administration. The composition will generally have a small particle size for example of the order of 5 microns or less. Such a particle size may be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the composition in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### Pharmaceutically Acceptable Salts

Pharmaceutically acceptable salts of the instant compounds, where they can be prepared, are also intended to be covered by this invention. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

### Administration Forms

The pharmaceutical composition may be prepared so it is suitable for one or more particular administration methods. Furthermore, the method of treatment described herein may involve different administration methods.

In general any administration method, wherein LAC may be administered to an individual in a manner so that active LAC may reach the site of disease may be employed with the present invention.

The main routes of drug delivery, in the treatment method are intravenous, oral, and topical, as will be described below. Other drug-administration methods, such as subcutaneous injection or via inhalation, which are effective to deliver the drug to a target site or to introduce the drug into the bloodstream, are also contemplated.

The mucosal membrane to which the pharmaceutical preparation of the invention is administered may be any mucosal membrane of the mammal to which the biologically active substance is to be given, e.g. in the nose, vagina, eye, mouth, genital tract, lungs, gastrointestinal tract, or rectum, preferably the mucosa of the nose, mouth or vagina.

Compositions of the invention may be administered parenterally, that is by intravenous, intramuscular, subcutaneous intranasal, intrarectal, intravaginal or intraperitoneal administration. The subcutaneous and intramuscular forms of parenteral administration are generally preferred. Appropriate dosage forms for such administration may be prepared by conventional techniques. The compositions may also be administered by inhalation, that is by intranasal and oral inhalation administration. Appropriate dosage forms for such administration, such as an aerosol formulation or a metered dose inhaler, may be prepared by conventional techniques.

The compositions according to the invention may be administered with at least one other compound. The compounds may be administered simultaneously, either as separate formulations or combined in a unit dosage form, or administered sequentially.

### Treatment

The composition according to the invention may be used for the manufacture of a medicament. Such medicament may be useful for the treatment of a variety of diseases wherein selective cytotoxicity is desirable. Apparently alpha-lactalbumin complexes are capable of preventing the attachment of bacteria and the spread of vira. The medicament may in an embodiment be used for the treatment of bacterial and/or viral infections. In further embodiments the medicament is for use in the treatment of cancer.

### Bacterial Infections

Apparently alpha-lactalbumin complexes are capable of preventing the attachment of bacteria to the cell surface, such as epithelial cells or by alternative means exerting a bactericidal effect on micro organisms.

Disease caused by bacterial infections include Anthrax, Bacterial Meningitis, Brucellosis, Campylobacteriosis, Cat Scratch Disease, Cholera, Diphtheria, Epidemic Typhus, Gonorrhea, Impetigo, Legionellosis, Leprosy (Hansen's Disease), Leptospirosis, Listeriosis, Lyme Disease, Melioidosis, MRSA infection, Nocardiosis, Pertussis (Whooping Cough), Plague, Pneumococcal pneumonia, Psittacosis, Q fever, Rocky Mountain Spotted Fever (RMSF), Salmonellosis, Scarlet Fever, Shigellosis, Syphilis, Tetanus, Trachoma, Tuberculosis, Tularemia, Typhoid Fever, Typhus; Urinary Tract Infections.

In an embodiment the composition according to the invention is for the treatment of bacterial infections.

Especially Streptococcus pneumoniae and Haemophilus influenzae are important causes of severe infections. In a preferred embodiment the composition according to the invention is for the manufacture of a medicament for the treatment of infections caused by such as S. pneumoniae or H. influenzae.

### Viral Infections

The selective cytotoxic activity of alpha-lactalbumin complexes may be directed against viral infected cells whereby the spread of virus is inhibited as cells are destroyed before the virus have multiplied.

Disease caused by viral infections include AIDS, AIDS Related Complex, Chickenpox (Varicella), Common cold, Cytomegalovirus Infection, Colorado tick fever, Dengue fever, Ebola haemorrhagic fever, Epidemic parotitis, Explodicitis, Hand, foot and mouth disease, Hepatitis, Herpes simplex, Herpes zoster, HPV, Influenza (Flu), Lassa fever, Measles, Marburg haemorrhagic fever, Infectious mononucleosis, Mumps, Poliomyelitis, Progressive multifocal leukencephalopathy, Rabies, Rubella, SARS, Smallpox (Variola), Viral encephalitis, Viral gastroenteritis, Viral meningitis, Viral pneumonia, West Nile disease, Yellow fever

In an embodiment the composition according to the invention is for the treatment of a viral infection.

Different types of viruses, such as viruses of the respiratory tract, gastrointestinal viruses, immunodeficiency viruses, such as HIV and viruses of the brain, such viral meningitis or virus of other internal organs may be treated using the composition according to the invention.

### Infection of the Respiratory Tract

Infections of the respiratory tract, e.g., meningitis, otitis and sinusitis are caused by bacteria which enter via the nasopharynx.

Viral infections of the respiratory tract may be caused by such as adenovirus, influenza viruses, respiratory cyncytial virus (RSV), parainfluenza, Phinoviruses and coronaviruses.

In an embodiment the composition according to the invention is for the treatment of infections of the respiratory tract. The medicament according to the invention may be inhaled in the form of a mist into the upper respiratory airways.

### Treatment of Tumors

In one embodiment tumors of both the benign or malignant type may further be treated using the composition according to the invention, based on the selective cytotoxic activity of alpha-lactalbumin complexes.

### Wart

A wart is generally a small, rough tumour, typically on hands and feet, that resembles a cauliflower. Warts are common, and are caused by a viral infection, specifically by the human papillomavirus (HPV). They typically disappear after a few months but can last for years and can recur.

A range of different types of wart have been identified, which differ in shape and site affected, including:

Common wart (verruca vulgaris): a raised wart with roughened surface, most common on hands and knees.

Flat wart (verruca plana): a small, smooth flattened wart, tan or flesh coloured, which can occur in large numbers; most common on the face, neck, hands, wrists and knees.

Filiform or digitate wart: a thread- or finger-like wart, most common on the face, especially near the eyelids and lips.

Plantar wart (verruca, verruca pedis): a hard sometimes painful lump, often with multiple black specks in the centre; usually only found on pressure points on the soles of the feet.

Mosaic wart: a group of tightly clustered plantar-type warts, commonly on the hands or soles of the feet.

Genital wart (venereal wart, condyloma acuminatum, verruca acuminata): wart affecting the genital areas.

In a preferred embodiment the composition according to the invention is for the treatment of warts, which is preferably treated by topical application of a medicament according to the invention.

### Papillomas

Papilloma refers to a benign epithelial tumor, which may or may not be caused by Human papillomavirus. Alternative causes are such as Choroid plexus papilloma (CPP).

Two types of papilloma often associated with HPV are squamous cell papilloma and transitional cell papilloma (also known as “bladder papilloma”.)

In a preferred embodiment the monomeric alpha-lactalbumin complex, preferably LAC composition according to the invention is for the treatment of papillomas, which is preferably treated by topical application of a medicament according to the invention.

### Cancer

Cancerous diseases are scientifically designated neoplasia or neoplasms and may be benign or malignant. Cancers are classified by the type of cell that resembles the tumor and, therefore, the tissue presumed to be the origin of the tumor. The following general categories are applied:

Carcinoma: malignant tumors derived from epithelial cells. This group includes the most common cancers, comprising the common forms of breast, prostate, lung and colon cancer.

Lymphoma and Leukemia: malignant tumors derived from blood and bone marrow cells.

Sarcoma: malignant tumors derived from connective tissue, or mesenchymal cells

Mesothelioma: tumors derived from the mesothelial cells lining the peritoneum and the pleura.

Glioma: tumors derived from glia, the most common type of brain cell

Germinoma: tumors derived from germ cells, normally found in the testicle and ovary.

Choriocarcinoma: malignant tumors derived from the placenta.

In a preferred embodiment the monomeric alpha-lactalbumin complex, preferably LAC composition according to the invention is for the treatment of cancer.

Medicaments for treatment of cancer are according to the invention preferably applied directly to the tumour.

### Mucosal Tumors

The conditions found at mucosal surfaces can be quite unique in terms of properties such as p.H. and the like. Mucosal surfaces are found inter alia in the nasal passages, in the mouth, throat, oesophagus, lung, stomach, colon, vagina and bladder.

Particular mucosal surfaces that may be treated with in accordance with the invention include throat, lung, colon and bladder surfaces which tumours.

### Bladder Cancer

Bladder cancer refers to any of several types of malignant growths of the urinary bladder. The most common type of bladder cancer begins in cells lining the inside of the bladder and is called urothelial cell or transitional cell carcinoma (UCC or TCC).

In a more preferred embodiment the monomeric alpha-lactalbumin complex, preferably LAC composition according to the invention is for the treatment of bladder cancer.

### Glioblastome

A glioma is a type of primary central nervous system (CNS) tumor that arises from glial cells. The most common site of involvement of a glioma is the brain, but they can also affect the spinal cord, or any other part of the CNS, such as the optic nerves.

In a more preferred embodiment the monomeric alpha-lactalbumin complex, preferably LAC composition according to the invention is for the treatment of glioma/glioblastome.

### Anaiogenesis.

Tumor angiongenesis is the proliferation of a network of blood vessels that penetrates in to cancerous growths, supplying nutrients and oxygen and removing waste products. The process of angiogenesis is initiated when tumor cells release molecules signalling to the normal host tissue, activating genes and proteins to encourage growth of new blood vessels. A series of natural inhibitors of angiogenesis have been identified, and are believed to prevent and/or inhibit the growth and spread of cancer cells.

The finding that alpha-lactalbumin complexes may inhibit angiogenesis further spread the applicability of alpha-lactalbumin in treatment and/or inhibition of cancer.

In an embodiment the monomeric alpha-lactalbumin complex, preferably LAC composition according to the invention is for inhibition of angiogenesis.

### Method of Treatment

An aspect of the invention relates to a method of treatment of an individual in need thereof comprising:
administering a medicament comprising:
   i. a composition comprising monomeric alpha-lactalbumin complex, preferably LAC, which is complex of alpha-lactalbumin and a fatty acid or a lipid, said alpha-lactalbumin being bovine or human alpha-lactalbumin of SEQ ID NO:1 or SEQ ID NO:2 or a functional equivalent thereof, wherein the composition comprises at least 95% by weight of monomeric alpha-lactalbuminin complex, preferably LAC and
   ii. pharmaceutical excipients.

According to the invention an individual in need is any individual suffering or at risk of acquiring any of the above mentioned diseases. Thus the treatment according to the invention may both be a curative treatment and/or a prophylactic treatment.

### Dosing Regimes

The dosage requirements of monomeric alpha-lactalbuminin complex, preferably LAC to be administered will vary with the particular drug composition employed, the route of administration and the particular subject being treated. Ideally, a patient to be treated by the present method will receive a pharmaceutically effective amount of the compound in the maximum tolerated dose, generally no higher than that required before drug resistance develops.

For all methods of use disclosed herein for the compounds, the daily oral dosage regimen will preferably be from about 0.01 to about 80 mg/kg of total body weight. The daily parenteral dosage regimen may be about 0.001 to about 80 mg/kg of total body weight. The daily topical dosage regimen will preferably be from 0.1 mg to 150 mg, administered one to four, preferably two or three times daily. The daily inhalation dosage regimen will preferably be from about 0.01 mg/kg to about 1 mg/kg per day. It will also be recognized by one of skill in the art that the optimal quantity and spacing of individual dosages of a compound or a pharmaceutically acceptable salt thereof will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a compound or a pharmaceutically acceptable salt thereof given per day for a defined number of days, can be ascertained by those skilled in the art using conventional course of treatment determination tests.

The daily dose of the active compound varies and is dependant on the type of administrative route, but as a general rule it is 1 to 100 mg/dose of active compound at personal administration, and 2 to 200 mg/dose in topical administration. The number of applications per 24 hours depend of the administration route, but may vary, e.g. in the case of a topical application in the no. se from 3 to 8 times per 24 hours, i.e., depending on the flow of phlegm produced by the body treated in therapeutic use.

The term “unit dosage form” as used herein refers to physically discrete units suitable as unitary dosages for human and animal subjects, each unit containing a predetermined quantity of a compound, alone or in combination with other agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host. The dose administered should be an “effective amount” or an amount necessary to achieve an “effective level” in the individual patient.

Since the “effective level” is used as the preferred endpoint for dosing, the actual dose and schedule can vary, depending on individual differences in pharmacokinetics, drug distribution, and metabolism. The “effective level” can be defined, for example, as the blood or tissue level desired in the patient that corresponds to a concentration of one or more compounds according to the invention.

### DETAILED DESCRIPTION OF THE DRAWINGS

FIG. 1A. Sequence alignments of equine, porcine, camelide, human, bovine and caprine alpha-lactalbumin. 1B: Sequence alignments of human and bovine alpha-lactalbumin.

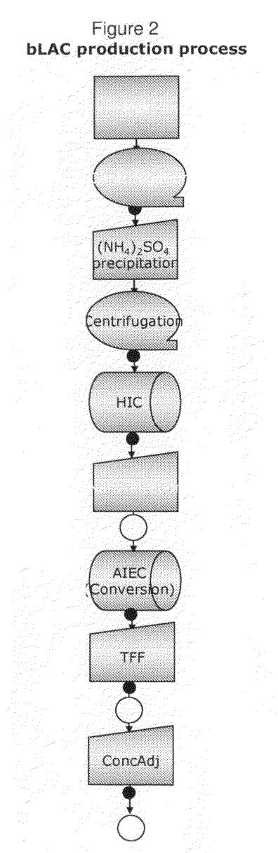

FIG. 2. Schematic overview of production process.

FIG. 3. Size Exclusion Chromatography (SEC) chromatograms of alpha-lactalbumin composition before (A) and after (B) conversion to the alpha-lactalbumin complex. (A) SEC-HPLC chromatograms of bLA purified from 4 consecutive HIC runs with a bLA load of above 40 mg/cm² resin showing a single monomeric bLA peak with a retention time of 24.6 minutes. B) SEC-HPLC chromatograms of bLAC produced from a pool of the purified bLA shown in A.

FIG. 4

Western blot of bLA and bLAC. Lane 1: Presicion Plus Merker (BioRad), Lane 2: Purified bLA (Fluka 61289), Lane 3-5 bLA purified as described in example 1, Lane 6-8 bLA purified with a load of 32 mg/cm² resulting in a dimer peak eluting at 23 min as shown in example 3—Lane 6 peak fraction eluting at 23 minutes, Lane 7 intermediate fraction, Lane 8 peak fraction eluting at 24.6 minutes, Lane 9 bLAC product stored at 4° C., Lane 10 bLAC product stored at −20° C. Monomeric bands migrate with an apparent molecular mass of only approximately 10 kDa and dimer bands as 15 kDa. A monomeric bLA sample has been analysed by MALDI-MS on a Bruker Microflex equipment, using a standard protocol, as having a mass of 14,196 Da as expected for a monomeric bLA. The dimer band migrates as having an apparent molecular mass of 28 kDa determined by linearity testing using SEC marker run with molecules of known molecular masses.

FIG. 5. Overview of cytotoxicity test.

FIG. 6 A Cytotoxicity test of human alpha-lactalbumin (hLAC) composition N177-58B, measured by luminescence (ViaLight) and trypan blue exclution. B Cytotoxicity test of bovine alpha-lactalbumin complex (bLAC) composition N262-34B measured by luminescence (ViaLight).

FIG. 7 A. Cytotoxicity test of alpha-lactalbumin complex (hLAC) composition N177-58B, and bovine alpha-lactalbumin complex (bLAC) composition N262-01E. FIG. 7 B. Cytotoxicity test of bovine alpha-lactalbumin (bLAC) composition N262-35B-093 and N262-35B-094.

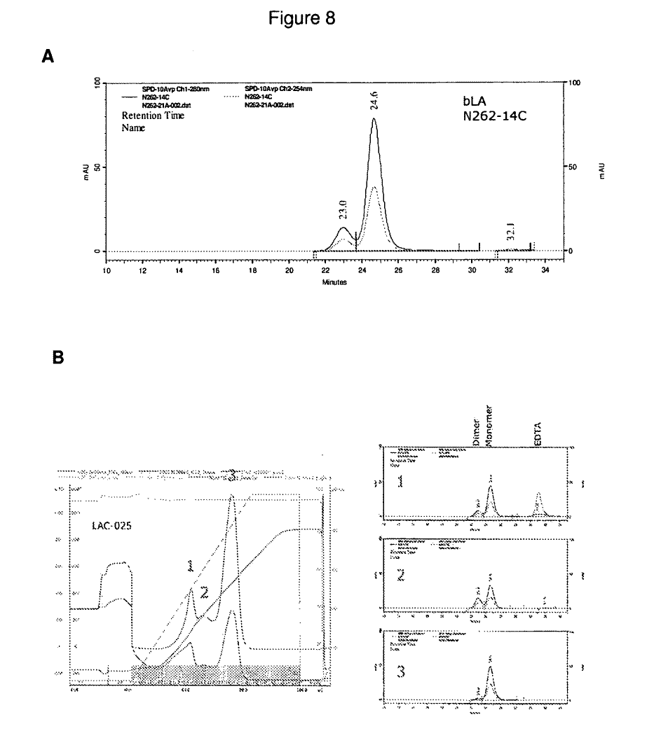

FIG. 8. SEC-HPLC chromatograms of bLA (A) purified from bovine milk with a load of 32 mg/cm² and the resulting bLAC product (B) following conversion. Both bLA and bLAC product show a dimer peak eluting at 23 minutes whereas monomeric bLA and bLAC elutes at 24.6±0.5 minutes. The samples are analysed by western blotting (FIG. 4).

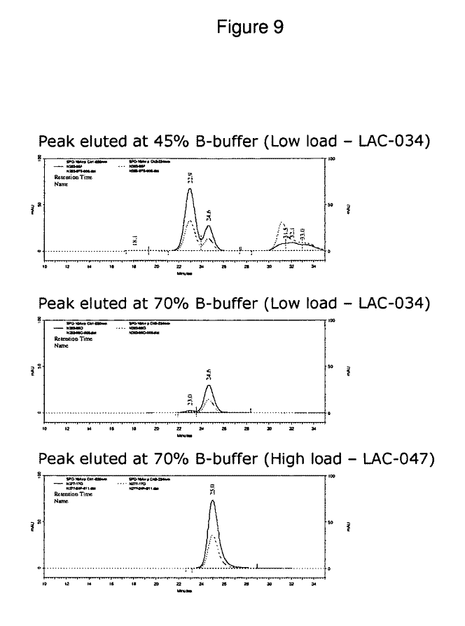

FIG. 9. Characterization of peaks eluted at different salt concentration during conversion of bLA to bLAC. Retention time of bLA/bLAC dimer=23 min; Retention time of bLA/bLAC monomer=25 min.

### EXAMPLES

### Example 1

### Purification of Bovine Alpha-Lactalbumin and Conversion to bLAC

Bovine whole milk (2 L) was defatted by centrifugation at 3500×g for 30 min. The defatted milk was ammonium sulphate precipitated overnight (264 g/L=40-45% saturation) after which the precipitate was centrifuged for 30 min at 3500×g. The ammonium sulphate precipitation supernatant was harvested and filtered first through a paper filter to remove any remaining precipitate or fat followed by a Millipore Optiscale filter with Polysep II media 1.0/0.5 μm.

The ammonium sulphate precipitation supernatant was conditioned for HIC chromatography by addition of 32.55 mL EDTA (0.25M)+24.97 mL Tris-EDTA (Tris 50 mM, EDTA 1 mM, pH 7.5)+67.45 mL per 100 mL ammonium sulphate precipitation supernatant. The conditioned media was adjusted to pH 7.5.

The bovine alpha-lactalbumin was purified using hydrophobic interaction chromatography by applying the conditioned media on to a GE Healthcare XK50/30 column packed with 300 mL phenyl Sepharose 6 FF High Sub (GE Healthcare) resin with a bed height of 15 cm and an area of 19.6 cm². The entire chromatographic run was performed with a flow rate of 40 cm/hr and load of 40 mg bLA per cm².

Before application of the conditioned media, the column was equilibrated with 5 column volumes (CV) of equilibration solution (Tris 50 mM, EDTA 1 mM, pH 7.5).

The column equilibration was followed by the application of the conditioned ammonium sulphate precipitation supernatant, after which the column was washed with 3 CV of equilibration solution (Tris 50 mM, EDTA 1 mM, pH 7.5). The bLA bound to the resin was eluted using 2 CV of elution solution (Tris 50 mM, CaCl₂ 1 mM, pH 7.5). The eluted bLA was collected with a fraction collector with a main peak sample volume of approximately 150-200 mL. The recovery of bLA was approximately 600 mg.

After the chromatographic run, the column was washed with water and regenerated with NaOH (1M).

The chromatographic run from equilibration until regeneration lasted approximately 415 minutes.

The bLA recovered from the HIC purification was filtrated through a Mini kleenpak 20 filter (Pall) before conversion.

The conversion of the alpha-lactalbumin was done on a GE Healthcare XK50/20 column packed with 200 mL DEAE Trisacryl plus M (Pall Biosepra) resin with a bed height of 10 cm and an area of 19.6 cm². The entire chromatographic run was performed with a flow rate of 40 cm/hr and a load of 15 mg bLA per cm².

Before application of the bLA recovered from the first HIC run, the column was preconditioned with oleic acid. First the column was equilibrated using 3 CV of equilibration solution (Tris 10 mM, NaCl 0.1M, pH 8.5). The oleic acid pre-conditioning was performed by applying 160 mL of a solution of 200 L oleic acid with 2.5 mL ethanol and 200 mL 10 mM Tris (pH 8.5) onto the column after which, the column was washed with 2 CV of equilibration solution (Tris 10 mM, NaCl 0.1M, pH 8.5) and then 3 gradient step elutions. Step 1 from 0-15% of elution solution (Tris 10 mM, NaCl 1 M, pH 8.5) for 1 CV, step 2 at 15% of elution solution (Tris 10 mM, NaCl 1 M, pH 8.5) for 2 CV and then 2 CV of 100% elution solution (Tris 10 mM, NaCl 1 M, pH 8.5). After the step elution, the column was equilibrated with 3 CV of equilibration solution (Tris 10 mM, NaCl 0.1 M, pH 8.5).

After preconditioning of the column with the oleic acid, the bLA obtained by HIC was conditioned by addition of 50 mL Tris (100 mM)+50 mL EDTA (0.25 M) and 300 mL Milli-Q H₂O per 100 mL HIC recovery, pH 8.5 and conductivity 6.0±0.5 mS/cm before it was applied onto the column.

Following application of the conditioned bLA containing HIC recovery, the column was washed with 2 CV of equilibration solution (Tris 10 mM, NaCl 0.1M, pH 8.5) and then 3 gradient step elutions. Step 1 from 0-15% of elution solution (Tris 10 mM, NaCl 1 M, pH 8.5) for 1 CV, step 2 at 15% of elution solution (Tris 10 mM, NaCl 1 M, pH 8.5) for 2 CV and then 4 CV of 100% elution solution (Tris 10 mM, NaCl 1 M, pH 8.5).

The bLAC was collected with a fraction collector with a main peak sample volume of approximately 150-200 mL. The recovery of bLAC was approximately 160 mg.

After each batch of bLA conversion, the column was regenerated with 2CV of acetic acid (1M), 2 CV of 100 mM Tris (pH 8.5), 2CV of NaOH (0.5 M), 4 CV of 100 mM Tris (pH 8.5) and 2-3 CV of 20% ethanol in which the column was stored.

The conversion chromatographic run from pre-conditioning equilibration until regeneration lasted approximately 560 minutes.

The bLAC was filtrated using a Mini Kleenpak 20 filter (Pall) before buffer change and concentration.

The converted bovine alpha-lactalbumin was buffer changed and concentrated using an Äkta CrossFlow equipment. The filtration was performed using a KvickStart 5 kDa ultrafiltration membrane. The retentate flow rate was 80 mL/min, a TMP limit of 4.0 bar and a load<9.6 mg/cm². The converted bovine alpha-lactalbumin was concentrated to approximately 9 mg/mL and buffer changed 7 times into 0.9% NaCl.

Determination of Monomeric and/or Multimeric bLAC by Western Blotting

The monomeric/multimeric composition of bovine alpha-lactalbumin in complex with oleic acid (bLAC) can be visualised by western blotting. The monomeric and multimeric forms of bLAC can be separated on a 16% SDS gel (PAGEgel) and subsequent blotting onto a PVDF membrane using standard protocols recommended by supplier (PAGEgel). The monomeric and multimeric forms can be visualised by immunodetection on the membrane using a HRP-labelled goat anti-bovine-lactalbumin (Bethyl A10-128P) diluted 1:6000 and detected with a chemiluminescent substrate (SuperSignal West Pico Chemiluminescent Substrate, Pierce) (See FIG. 3)

Determination of Monomeric and/or Multimeric bLAC by SEC-HPLC

The monomeric/multimeric composition of bovine alpha-lactalbumin in complex with oleic acid (bLAC) can be visualised by SEC-HPLC. Monomeric and multimeric forms of bLAC can be separated by injecting 25 μL bLAC onto a Superdex 75, Tricorn 10/300 column (GE Health Care). The column is equilibrated with elution buffer (Tris 10 mM, NaCl 140 mM, NaN₃ 0.001%, pH7.4) until a stable baseline is obtained. Following application of the bLAC sample, the sample is eluted with 40 mL elution buffer (Tris 10 mM, NaCl 140 mM, NaN₃ 0.001%, pH7.4) at a flow rate of 38 cm/hr resulting in a runtime of 80 minutes per sample. The monomeric form of bLAC elutes after 24.6±0.5 min whereas the dimer elutes after 23±0.5 minutes (See FIG. 2).

### Example 2

### Cytotoxicity of Monomeric Alpha-Lactalbumin Composition

The cytotoxic activity of alpha-lactalbumin compositions is tested in a viability assay following the outline below and shown in FIG. 4 by use of the ViaLight® PLUS Cell Proliferation and Cytotoxicity BioAssay Kit from Cambrex. The potency of LAC preparations was determined from their ability to kill the murine lymphocytic leukaemia cell line L1210 (ATCC cat. no CCL-219) cultivated in RPMI 1640 medium (without HEPES) supplemented with 5% fetal bovine serum, 1% non-essential amino acids and 2 mM sodium pyruvate.

From each tested LAC preparation a suitable dilution series was made in 0.9% NaCl solution. 20 μL of each dilution was in triplicate mixed with 50 μL of cell suspension containing 100,000 or 200,000 PBS-washed L1210 cells in RPMI 1640 medium without serum and HEPES in a 96-well white-walled cell culture plate. After 1 hour incubation at 37° C. and 5% CO₂ 5 μL fetal bovine serum was added per well to inactivate all extracellular LAC. Another 1 hour incubation at 37° C. and 5% CO₂ allowed cells to further undergo apoptosis, and the viability of the cultures was then determined.

The viability of the cultures can be determined by trypan blue exclusion or by determining the amount of ATP present relative to control cultures treated with 20 μL 0.9% NaCl solution instead of LAC. ATP is present in all metabolically active cells. In dead cells ATP quickly disappears and thus, it can be assumed that only viable cells contain ATP. Therefore, there is a direct correlation between the relative level of ATP and the percentage of viable cells. The relative amount of ATP is determined using the ViaLight PLUS kit from Cambrex and a luminometer. To quantify ATP the following reaction is used:

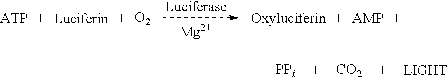

Light emission is measured as luminescence on a luminometer. The luminescence is linearly related to the ATP concentration. When using this method the plate was removed from the incubator and allowed to cool to room temperature for 10 minutes. 50 μL of lysis reagent from the ViaLight PLUS kit was added per well and the plate is incubated for 10 minutes at room temperature. Then 100 μL AMR PLUS solution from the ViaLight PLUS kit was added to each well, and the plate was incubated for 5 minutes at room temperature. The plate was read in a luminometer with an integrated reading time of 1 second.

From the resulting data a graph of LAC dose (μg per 100,000 cells or pg per cell) versus viability or luminescence can be plotted and the LD50 (The dose of LAC that is lethal to 50% of the L1210 cells under the conditions and specific exposure time used) can be determined. The result from the luminescence measurement method is found to be equivalent to results obtained by trypan blue exclusion.

Results obtained testing several batches of alpha-lactalbumin are shown in the table here below.

TABLE 1
Cytotoxic activity towards L1210 cells.

| | LD50 dose at the given number of L1210 cells in 70 μL | | | | | | |
|---|---|---|---|---|---|---|---|
| | μl/ | | pg/cell at | | pg/cell at | mg/ml at | mg/ml at |
| alpha- | 100.000 | μg/100.000 | 100.000 | μg/200,000 | 200,000 | 100.000 | 200,000 |
| lactalbumin | cells | cells | cells | cells | cells | cells | cells |
| bLAC | 0.62 | 1.6 | 16 | | | 0.023 | |
| N262-34B | | | | | | | |
| (4-fold | | | | | | | |
| dilution of | | | | | | | |
| N262-33C) | | | | | | | |
| (contains | | | | | | | |
| 2.6 mg/ml) | | | | | | | |
| bLAC | | 5.8 | 58 | | | 0.083 | |
| N262-01E | | | | | | | |
| (contains | | | | | | | |
| 9.7 mg/ml) | | | | | | | |
| bLAC | | 1.4 | 14 | | | 0.020 | |
| N262-35B- | | | | | | | |
| 093 0g 094 | | | | | | | |
| (contains | | | | | | | |
| 9.1 mg/ml) | | | | | | | |
| hLAC | | 9.8-11.4 | 98-114 | 17.4 | 87 | 0.14-0.16 | 0.25 |
| N177-58B | | | | | | | |
| (assuming | | | | | | | |
| 100% | | | | | | | |
| hLAC) | | | | | | | |
| (control) | | | | | | | |

### Example 3

### Load of HIC as a Determinant for the Monomeric/Multimeric bLAC Ratio of the Alpha-Lactalbumin Complexes

The monomeric/multimeric bLAC ratio may be controlled by the monomeric/multimeric composition of the bLA purified by the hydrophobic interaction chromatography as described in example 1.

A load of 2 mg/mL gel (32 mg/cm²) results in a bLA with both dimer and monomer forms (FIG. 8) whereas a load of 6 mg/mL resin (90 mg/cm²) result of monomeric bLA only (example 1, FIG. 3). The conversion of bLA was performed as described in example 1.

### Example 4

An oleic acid solution of 3.5 mM was freshly prepared prior each sample conditioning. Oleic acid (20 μL) was mixed with Ethanol 96% (0.25 mL) and Tris-HCl (10 mM) pH 8.5, NaCl (0.1 M) (20 mL).

bLA purified by hydrophobic interaction chromatography (HIC) was conditioned with EDTA, Tris (0.1 M pH 8.5) and oleic acid solution. A description of the different samples conditioning are shown in Table 4.

TABLE 2
bLA sample conditioning

| | | | Tris pH 8.5 | EDTA | Oleic acid | | Final |
|---|---|---|---|---|---|---|---|
| Run ID | bLA ID | bLA | (1 M) | (0.25 M) | (3.5 mM) | MilliQ H2O | Volume |
| LAC- | N262-26D | 9 mL | 3.6 mL | 0.144 mL | 20.6 mL | 2.7 mL | 36 mL |
| 034 | 2.91 mg/mL | (26 mg) | | (1 mM) | (2 mM) | | |
| | | (1.8 μmole) | | (36 μmole) | (72 μmole) | | |
| LAC- | N262-54D | 6.2 mL | 2.48 mL | 0.099 mL | 14.2 mL | 1.86 mL | 25 mL |
| 035/ | 4.03 mg/mL | (25 mg) | | (1 mM) | (2 mM) | | |
| 037/ | | (1.8 μmole) | | (24.8 μmole) | (49.6 μmole) | | |
| 041 | | | | | | | |
| LAC- | N262-54D | 9 mL | 3.6 mL | 0.144 mL | 20.6 mL | 2.7 mL | 36 mL |
| 042 | 4.03 mg/mL | (36 mg) | | 1 mM | 2 mM | | |
| | | (2.5 μmole) | | (36 μmole) | (72 μmole) | | |
| LAC- | N262-54D | 18 mL | 7.2 mL | 0.288 | 41.1 mL | 5.4 mL | 72 mL |
| 038/ | 4.03 mg/mL | (72 mg) | | 1 mM | 2 mM | | |
| 047 | | (5.0 μmole) | | (72 μmole) | (144 μmole) | | |

Prior to application on the AIEC column the conditioned samples were mixed for approximately 30 min. at room temperature.

A column was newly packed with Q Sepharose XL resin (GE healthcare). The different bLA samples shown in Table 2 were applied on this column. The chromatographic parameters for the AIEC runs were the following.

| | | |
|---|---|---|
| Column | Tricorn 10/100 with 8 mL packed with Q Sepharose | |
| | XL (GE HealthCare #17-5072-99) | |
| bLA | LAC-034: 34 mL conditioned sample (31 mg/cm²) | |
| loads | LAC-035/041: 23 mL conditioned sample (30 mg/cm²) | |
| | LAC-042: 33 mL conditioned sample (42 mg/cm²) | |
| | LAC-047: 70 mL conditioned sample (90 mg/cm²) | |
| bLAC | Wash: Equilibration solution | (2 CV) |
| Elution | | |
| profile | | |
| Step | Gradient step 1: Step 45% solvent B (in Solvent A) | (2CV) |
| gradient | Gradient step 2: Step 70% solvent B (in Solvent A) | (2CV) |
| | Gradient step 3: Step 100% solvent B | (2CV) |

Several regeneration strategies were tested:

| | | |
|---|---|---|
| Regeneration | Acetic acid (1M) | (2CV) |
| Before LAC034/035 | Wash with washing solution | (2CV) |
| | NaOH (0.5M) | (2CV) |
| | Wash with washing solution | (2CV) |
| | Ethanol (20%) | (2CV) |
| Regeneration | Acetic acid (1M) | (2CV) |
| Before | Wash with washing solution | (2CV) |
| LAC041/042/047 | NaOH (0.5M) | (2CV) |
| | Wash with washing solution | (2-10CV) |
| | Ethanol (20%) | (2CV) |
| | Ethanol (70%) | (2CV) |
| | Ethanol (20%) | (2CV) |
| Solvent A | Tris (10 mM) | |
| | NaCl (0.1M) | |
| | pH = 8.5 (22° C.) | |
| Solvent B | Tris (10 mM) | |
| | NaCl (1M) | |
| | pH = 8.5 (22° C.) | |

The regeneration procedure with 70% ethanol is applied in order to remove hydrophobically bound substances like oleic acid or bLAC.

### Yield and Potency

The yields of the conversion runs were determined by size exclusion HPLC (SE-HPLC) run according to standard procedures.

The potency of the converted bLA was determined by cell killing. Cell killing assays were performed as described herein below in Example 5. The potency of bLAC determined by cell killing assay is given in pg bLAC per cell (LD50). Before testing in cell killing assay, the bLAC solutions were desalted against NaCl (0.9%) using NAP-25 desalting column (GE HealthCare).

### Results: Comparison of the Conversion Runs

The chromatograms of several conversion runs on Q sepharose XL are seen in FIG. 9. In all the runs, the final concentration of EDTA was 1 mM.

The results of the conversion runs are resumed in Table 3. The yield calculations and the cell killing abilities were performed on the peak eluted at 70% solvent B (in Solvent A).

TABLE 3
Summary of the conversion runs

| | | | | | SE- | Purity SE- | |
|---|---|---|---|---|---|---|---|
| | | Load | Yield | Yield | HPLC | HPLC | Killing Assay |
| Prod. ID | Item no.* | (mg/cm²) | (mg/cm²) | (%) | (mg/mL) | (%) Monomer | LD50 (pg/cell)¹ |
| LAC-034 | N263-95G (96B) | 31 | 26 | 84 | 3.3 | 93 | 32 |
| LAC-035 | N263-99E (99G) | 30 | 25 | 83 | 3.3 | 100 | 24 |
| LAC-041 | N277-07C (07E) | 30 | 28 | 93 | 3.2 | 100 | 21 |
| LAC-042 | N277-09F (09G) | 42 | 38 | 90 | 4.0 | 100 | 27 |
| LAC-047 | N277-17G (18A) | 90 | 76 | 84 | 8.4 | 100 | 42 |

*In bracket the sample ID after desalting used for cell killing assay
**Conversion with 0.2 mM oleic acid

From the results showed in Table 3, it can be seen that the yield of the conversion is over 80%.

Three different bLA loads 30, 42 and 90 mg/cm² corresponding to 2.9, 4.1 and 8.8 mg/mL Q sepharose XL were tested in LAC-041, -042 and -047. The yield of the conversion was found still over 80% at the highest load (LAC-047).

### SE-HPLC

The peaks eluted from the different conversion runs of bLA to bLAC were analyzed by SE-HPLC. These analyses were used for the quantification of bLA/bLAC before and after conversion in order to determine the yield of the runs (cf. Table 3). This analysis gives also an indication of the purity of bLAC after conversion, i.e. monomer, dimer, or aggregates forms. This is particularly important when the conversion run were performed at different bLA loads.

When looking at the SE-HPLC chromatogram (FIG. 9), it is seen that the bLAC recovered at 70% B-buffer was mainly in monomer form. There was a tendency that at higher load, only bLAC monomer was recovered. The bLA dimer was then recovered in the first step gradient at 45% B-buffer, with bLA monomer in lower amount. No cell killing activity was recovered in the 45% B-buffer elution.

1. A pharmaceutical composition comprising a monomeric alpha-lactalbumin complex of alpha-lactalbumin and a fatty acid or a lipid, said alpha-lactalbumin being bovine alpha-lactalbumin or a functional equivalent thereof, wherein the composition comprises at least 95% by weight of monomeric alpha-lactalbumin.

2. The composition according to claim 1, wherein the composition comprises at least 98% by weight of monomeric alpha-lactalbumin.

3. The composition according to claim 1, wherein the amount of multimeric or oligomeric alpha-lactalbumin in the composition is below the level of detection by methods such as PAGE and immunoblotting.

4. The composition according to claim 1, wherein the cytotoxic activity measured as LD50 is less 0.1 mg/ml.

5. The composition according to claim 1, wherein the cytotoxic activity measured as LD50 is less than 0.04 mg/ml.

6. The composition according to claim 1, wherein the fatty acid or lipid is mono-saturated acid selected from the group of: C16:1:6cis and trans, C16:1:9cis and trans, C16:1:11cis and trans, C18:1:6cis or trans, C18:1:9cis and trans, C18:1:11cis or trans, C18:1:13cis or trans, C20:1:9 cis and trans, C20:1:11cis and trans, C20:1:13cis and trans.

7. The composition according to claim 1, wherein the functional equivalent is at least 70% identical to bovine alpha-lactalbumin.

8. The composition according to claim 1, wherein the functional equivalent comprise at least one consecutive sequence of bovine alpha-lactalbumin of at least 40 amino acids.

9. The composition according to claim 1, wherein the functional equivalent comprise sequence at least two amino acid segments of bovine alpha-lactalbumin selected from the group of; amino acid 4-8, 34-38, 48-58, 75-88, 91-97 and amino acid 103-121 of bovine alpha-lactalbumin.

10. The composition according to claim 1, wherein the functional equivalent of alpha-lactalbumin is selected from the group of; equine, caprine, bovine, camelide and porcine alpha-lactalbumin.

11. The composition according to claim 1, wherein the alpha-lactalbumin is bovine.

12. The composition according to claim 1, wherein the protein concentration is more than 5 mg/ml.

13. A method of producing a pharmaceutical composition comprising monomeric alpha-lactalbumin complex of alpha-lactalbumin and a fatty acid or a lipid, said alpha-lactalbumin being bovine alpha-lactalbumin or a functional equivalent thereof, wherein the composition comprises at least 95% by weight of monomeric alpha-lactalbumin comprising the steps of:
a. obtaining an alpha-lactalbumin composition comprising at least 95% by weight of monomeric alpha-lactalbumin,
b. conversion of said alpha-lactalbumin to alpha-lactalbumin complex
i. by release of calcium from said alpha-lactalbumin and
ii. binding of fatty acid or lipid to said alpha-lactalbumin, and
c. purification of the alpha-lactalbumin complex.

14. A method of treating respiratory tract infection, warts, papiloma, cancer, bladder cancer or angiogenesis comprising administering a composition of claim 23 to a person in need of said treatment.

15. (canceled)

16. (canceled)

17. (canceled)

18. (canceled)

19. (canceled)

20. (canceled)

21. (canceled)

22. A composition comprising a monomeric alpha-lactalbumin complex of alpha-lactalbumin and a fatty acid or a lipid, said alpha-lactalbumin being bovine alpha-lactalbumin or a functional equivalent thereof, wherein the composition comprises at least 95% by weight of monomeric alpha-lactalbumin.

23. The composition of claim 1, further comprising a pharmaceutical excipient.


SEQUENCE LISTING (4 sequences)

SEQ ID NO: 1 - protein, 123 aa, Bo taurus
EQLTKCEVFRELKDLKGYGGVSLPEWVCTTFHTSGYDTQAIVQNNDSTEYGLFQINNKIWCKDDQNPHSSNICNISCDKFLDDDLTDDIMCVKKILDKVGINYWLAHKALCSEKLDQWLCEKL

SEQ ID NO: 2 - protein, 123 aa, Homo sapiens
KQFTKCELSQLLKDIDGYGGIALPELICTMFHTSGYDTQAIVENNESTEYGLFQISNKLWCKSSQVPQSRNICDISCDKFLDDDITDDIMCAKKILDIKGIDYWLAHKALCTEKLEQWLCEKL

SEQ ID NO: 3 - protein, 142 aa, Bo taurus
MMSFVSLLLVGILFHATQAEQLTKCEVFRELKDLKGYGGVSLPEWVCTTFHTSGYDTQAIVQNNDSTEYGLFQINNKIWCKDDQNPHSSNICNISCDKFLDDDLTDDIMCVKKILDKVGINYWLAHKALCSEKLDQWLCEKL

SEQ ID NO: 4 - protein, 142 aa, Homo sapiens
MRFFVPLFLVGILFPAILAKQFTKCELSQLLKDIDGYGGIALPELICTMFHTSGYDTQAIVENNESTEYGLFQISNKLWCKSSQVPQSRNICDISCDKFLDDDITDDIMCAKKILDIKGIDYWLAHKALCTEKLEQWLCEKL